(19) United States
(12) Patent Application Publication
(10) Pub. No.: US 2011/0280799 A1
(43) Pub. Date: Nov. 17, 2011

(54) NANOCELLS FOR DIAGNOSIS AND TREATMENT OF DISEASES AND DISORDERS

(75) Inventors: Shiladitya Sengupta, Waltham, MA (US); Ram Sasisekharan, Bedford, MA (US); Carlos J. Bosques, Cambridge, MA (US); David A. Eavarone, North Quincy, MA (US); Pochi Shum, Cambridge, MA (US)

(73) Assignee: MASSACHUSETTS INSTITUTE OF TECHNOLOGY, Cambridge, MA (US)

(21) Appl. No.: 13/155,244

(22) Filed: Jun. 7, 2011

Related U.S. Application Data
(63) Continuation of application No. 11/886,283, filed on Sep. 25, 2008, now abandoned.
(60) Provisional application No. 60/661,627, filed on Mar. 14, 2005.
(60) Provisional application No. 60/708,012, filed on Aug. 12, 2005.

(51) Int. Cl. A61K 51/12; A61K 31/56; A61K 38/46; A61P 35/00; A61P 35/04; A61P 27/02; A61P 1/00; A61P 29/00; A61P 19/02; A61P 37/06; A61P 27/06; A61P 43/00; A61P 11/06; A61P 11/00; A61K 9/14; B82Y 5/00; B82Y 15/00

(52) U.S. Cl. 424/1.37; 424/498; 514/169; 424/94.6; 977/762; 977/773; 977/700; 977/906; 977/927

(57) ABSTRACT

The present invention relates to novel nanocell compositions and their use in imaging, diagnostic and treatment methods. In one embodiment, nanocells tailored for imaging methods comprise a nanocore surrounded by a lipid matrix, and are modified to contain a radionuclide core or a nanocore with an emission spectra. The nanocells may be size restricted such as being greater than about 60 nm so that they selectively extravasate at sites of angiogenesis (e.g. tumor) and do not pass through normal vasculature or enter non-tumor bearing tissue. In this way, angiogenic sites can be both detected and treated. In another embodiment, nanocells are tailored for various treatment methods, including the treatment of brain cancer, asthma, Grave's Disease, Cystic Fibrosis, and Pulmonary Fibrosis.

### CROSS REFERENCES TO RELATED APPLICATIONS

This application is a continuation of U.S. application Ser. No. 11/886,283, which is the National Stage of PCT/US06/09213, filed Mar. 14, 2006, which claims the benefit of U.S. Provisional Application No. 60/661,627, filed Mar. 14, 2005 and U.S. Provisional Application No. 60/708,012, filed Aug. 12, 2005. These applications are herein incorporated by reference in their entirety.

### FIELD OF THE INVENTION

The present invention relates to novel diagnostic agents, method for their use in imaging, such as identification of malignant cells, preferably solid tumor detection, and kits for preparing and using such diagnostic agents. Also encompassed are novel nanocell platforms for targeting cells, method for their use in treatment of diseases or disorders, and kits for preparing and using the same.

### BACKGROUND OF THE INVENTION

The ability to obtain in vivo images has assisted in treatment, diagnosis and prognosis of a variety of diseases and disorders. A range of imaging agents, for example radioimaging agents, have been developed, but have suffered from problems such as cost, complexity, and the need to identify specific ligands that target desired tissues.

A limitation of current diagnostic imaging methods is that it is often not possible to deliver the imaging agent specifically to the tissue or cell type that one wishes to image. What is needed is an agent that is specific for the target tissue, yet does not bind appreciably to surrounding non-target cells. In the area of diagnostic imaging of cancer, current methods for tumor-specific imaging are hindered by imaging agents that also accumulate in normal tissues. Cancer refers to a range of different malignancies and remains a major health concern. Despite increased understanding of many aspects of cancer, the methods available for its detection continue to have limited success. The ability to detect a malignancy as early as possible, and assess its severity, would be extremely helpful in designing an effective therapeutic approach. Thus, methods for detecting the presence of effective therapeutic approach. Thus, methods for detecting the presence of malignant cells and understanding changes in their disease state are desirable, and will contribute to our ability to tailor cancer treatment to a patient's disease.

Various radioactive metals (radionuclides) have been prepared including Tc, Ru, Co, Pt, Fe, Os, Ir, W, Re, Cr, Mo, Mn, Ni, Rh, Pd, Nb and Ta, see e.g., U.S. Pat. Nos. 4,452,774; 4,826,961, 5,783,170; 5,807,537; 5,814,297; 5,866,097; and U.S. Patent Application Publication No. 2002/187099. However, in order to effectively deliver such radionuclides one needs to prepare coordination complexes with ligands. The specific coordination requirements of particular radionuclides place constraints on the ligands that can be used, which in turn place limits on what are viable targets. Ideally, a radionuclide imaging complex should display specific targeting in the absence of substantial binding to normal tissues, and a capacity for targeting to the desired targets. For example, a variety of tumor types and at a variety of stages. Thus, there still exists a need in the art for methods to develop and achieve effective delivery of imaging agents to target sites such as tumors by simple and general means.

Tailored therapies for various diseases and disorders are also needed. Although numerous therapies currently exist for cancers, diabetes, asthma, cystic fibrosis, and other diseases and disorders, the actual results are not entirely satisfactory. One problem may be the presently available modes, dosage, and timing of delivery. For example, while anti-inflammatory therapy is a vital treatment for alleviating asthmatic attack, delivering an anti-inflammatory during an acute attack can be ineffective due to its inability to reach its target site. A fast-acting and small dose of bronchodilator administered first, followed by a more long-lasting anti-inflammatory, is desired. However, current therapies provide for a large dose corticosteroid and bronchodilator administered concurrently, which results in ineffective treatment and unwanted side effects due to unnecessarily large doses of pharmaceutical compounds. A composition and method that would permit better tailoring of dosing, timing and delivery in a single administration is needed. Also needed are convenient, small dose administrations, preferably single dose administrations, of combinations of drugs so as to attain better patient compliance, reduce healthcare costs and provide patients with a more personalized treatment plan.

### SUMMARY OF THE INVENTION

We have now discovered novel compositions and methods for detecting a desired target in vivo, and diagnosing and treating desired diseases and/or disorders, such as angiogenic diseases and disorders, e.g. tumors.

In one embodiment, novel nanocell compositions are disclosed for their use in imaging methods (“imaging nanocells” or “radionuclide nanocells”). Such imaging nanocells comprise a nanocore surrounded by a lipid matrix (see U.S. Patent Application No. 60/549,280, filed Mar. 2, 2004), and are modified to contain a radionuclide core or a nanocore with an emission spectra. In another embodiment, methods for detecting a desired target in vivo using the novel imaging nanocells is disclosed. In one preferred embodiment, the nanocells are size restricted such as being greater than about 60 nm so that they selectively extravasate at sites of angiogenesis (e.g. tumor) and do not pass through normal vasculature or enter non-tumor bearing tissue. Other sizes can be calculated for other conditions. Preferably, the nanocell containing radioimaging agents are used in solid tumor detection.

The radionuclide containing nanocells comprise an inner nanocore of radionuclide, and an outer nanoshell of lipid with associated PEG. The nanocell may also contain a quantum dot nanocore or a gandolinium or fluorochrome-conjugated nanoparticle, which can be excited using a defined wavelength and emits light at a defined wavelength. In one embodiment the nanocell can contain ligands that bind to specific targets such as organs, tissues, or cells. In one embodiment, the ligands could be peptides, carbohydrates, lipids or derivatives there-of, which can bind to carbohydrates, peptides or lipids on cell surface or their derivatives.

In a preferred embodiment of the present invention, the nuclear nanocore is about 60 nm to about 120 nm in total diameter. Alternatively, the nuclear nanocell may be from about 60 nm to about 600 nm in diameter.

A method for the detection of angiogenic diseases or disorders, in particular tumors, in vivo is encompassed in the present invention. In this method, an individual is administered a radionuclide nanocell of the present invention, which is size restricted to greater than about 60 nm.

A method for synthesizing the imaging composition of the present invention is also disclosed.

In one embodiment, the imaging nanocell further comprises a caged therapeutic that is released only when the nanocore is excited. Alternatively, the radiological diagnostic nanocell comprises a non-caged therapeutic.

In another embodiment, a targeting ligand is attached to the outer surface of the nanocell (i.e. on the PEG or lipid nanoshell) to further enhance and target delivery of the imaging agent to particular organs, tissue, or cells.

Various routes of administration of the imaging agent can be employed in the disclosed methods. In some embodiments, the radioimaging nanocell is administered via a route selected from the group consisting of peroral, intravenous, intraperitoneal, inhalation, and intratumoral.

The disclosed methods and compositions employ radiological imaging agents as disclosed herein for the detection, treatment and diagnosis of diseases and/or disorders such as cancer and angiogenic diseases and disorders.

In another embodiment, novel nanocells that are tailored (“tailored nanocells”) so that they directly and efficiently deliver appropriate therapies for appropriate lengths of time to relevant biological sites are disclosed. Methods for treating individuals with disease and/or disorders using these tailored nanocells are also encompassed.

In one preferred embodiment, the tailored nanocell is surface modified with a targeting moiety that delivers the nanocell to an appropriate biological site and may itself act as an effector, or modulator of, cellular function. The targeting moieties bind to specific targets such as organs, tissues, or cells. In one embodiment, the targeting moiety are peptides, carbohydrates, lipids or derivatives there-of, which can bind to carbohydrates, peptides or lipids on cell surface or their derivatives.

In general, the tailored nanocells of the present invention comprise an inner nanocore containing at least one first therapeutic and an outer nanoshell comprised of lipid, which contains at least one second therapeutic that differs from the first therapeutic. The nanoshell may also be associated poly-ethylene glycol (PEG) and a targeting moiety as described above. Alternatively, the nanocore may contain at least one therapeutic that is substantially similar to the at least one therapeutic contained in the nanoshell. In this embodiment, the composition of the matrix encapsulating the first therapeutic differs from the composition of the matrix encapsulating the at least one second therapeutic so that the therapies are released at different times and/or rates.

In one embodiment, methods for treating a desired disease or disorder, e.g. tumors, using the tailored nanocells of the present invention is disclosed. In this embodiment, the nanocell comprises a nanocore containing a first therapeutic that is selectively chosen so as to act over an extended period of time and a second therapeutic encapsulated within the outer nanoshell that is selectively chosen so as to act immediately and over a shorter period of time. In one preferred embodiment the tailored nanocells are size restricted such as being greater than about 60 nm so that they selectively extravasate at sites of angiogenesis (e.g. tumor, macular degeneration, rheumatoid arthritis, psoriasis, atherosclerosis, etc) and do not pass through normal vasculature or enter non-tumor bearing tissue. In a preferred embodiment of the present invention, the tailored nanocell is about 60 nm to about 120 nm in total diameter.

For example, an individual suffering from macular degeneration can have an anti-angiogenesis compound, such as, for example, Avastin™ or a vascular targeting agent such as combretastatin, delivered to the eye in combination with another therapy, such as, for example, alpha adrenergic agonists. In another embodiment, a composition and method for the treatment of brain tumors, such as, for example, gliomas, neuronal tumors, anaplastic glioma and meningioma are disclosed. In this embodiment, the tailored nanocell composition comprises a nanocore with a first therapeutic consisting of a corticosteroid and a nanoshell with a second therapeutic consisting of a chemotherapeutic. The corticosteroid may be selected from the group consisting of cortisol, cortisone, hydrocortisone, fludrocortisone, dexamethasone, prednisone, fluticasone, methylprednisonlone, or prednisolone etc. Likewise, the chemotherapeutic may be selected from the group consisting of nitrosurea-based chemotherapy such as, for example, BCNU (carmustine), CCNU (lomustine), PCV (procarbazine, CCNU, vincristine), or temozolomide (Temodar). Preferably, the first therapeutic is encapsulated in a biodegradable polymer such as PLGA at defined ratio, so as to provide for sustained or slow-release kinetics of the corticosteroid. The chemotherapeutic is also encapsulated in a biocompatible polymer at a specific ratio so as to provide for a more immediate but sustained release of the chemotherapeutic. The nanocell may also contain an anti-angiogenesis agent or a vascular targeting agent.

A method for the treatment of brain tumors utilizing the tailored nanocell composition is also disclosed. In this method, an individual is administered a tailored nanocell of the present invention systemically or by directly injecting it into the site in need. Preferably, the tumor is resected and the tailored nanocells are delivered to the area of resection at this time.

In another embodiment, a composition and method for the treatment of asthma is disclosed. In this embodiment, the tailored nanocell composition comprises a nanocore with a first therapeutic consisting of a corticosteroid and a nanoshell with a second therapeutic consisting of a bronchodilator. One can also add additional layers around the nanocell to further fine tune delivery of specific drugs. The corticosteroid may be selected from the group consisting of cortisol, cortisone, hydrocortisone, fludrocortisone, prednisone, methylprednisonlone, or prednisolone etc. The bronchodilator may be selected from the group consisting of an anticholinergic, such as ipratropium or a beta-agonist such as albuterol, metaproterenol, salmeterol, pirbuterol, or levalbuteral. The composition for the treatment of asthma allows for an individual to be administered a smaller dose of corticosteroid than is normally available because the bronchodilator in the nanoshell acts first to make available the biological sites of action for the corticosteroid. In one embodiment, the nanocore may comprise a biodegradable polymer such as PLGA and the nanoshell may comprise a water soluble carrier such as lactose. The size may be about 10² to about 10⁴ nm.

A method for the treatment of asthma utilizing this tailored nanocell composition is also disclosed. In one method, an individual is administered, via inhalation, a tailored nanocell of the present invention.

In another embodiment, a composition and method for the treatment of Grave's Disease is disclosed. In this embodiment, the tailored nanocell composition comprises a nanocore with a first therapeutic consisting of a iopanoic acid/ipodate sodium and a nanoshell with a second therapeutic consisting of an antithyroid drug such as, for example, methimazole, carbimazole, or propylthiouracil. Alternatively, the first therapeutic may be a radionuclide, such as iodine 123. Likewise, the second therapeutic, in the nanoshell, may also be a beta-blocker (i.e. propanolol). In another embodiment, the composition for the treatment of Grave's Disease may comprise more than one therapeutic in the nanocore and more than one therapeutic in the nanoshell.

A method for the treatment of Grave's Disease utilizing the tailored nanocell composition is also disclosed. In this method, an individual is administered a tailored nanocell of the present invention systemically via parenteral or enteral routes.

In another embodiment, a composition and method for the treatment of Cystic Fibrosis is disclosed. In this embodiment, the tailored nanocell composition comprises a nanocore with at least one first therapeutic consisting of an antibiotic. In addition to an antibiotic, the core may also contain an optional bronchodilator or steroid. In this embodiment, the nanoshell contains at least one second therapeutic consisting of recombinant human deoxyribonuclease (rhDNase).

A method for the treatment of Cystic Fibrosis utilizing the tailored nanocell composition is also disclosed. In this method, an individual is administered a tailored nanocell of the present invention via inhalation.

In another embodiment, a composition and method for the treatment of idiopathic pulmonary fibrosis is disclosed. In this embodiment, the tailored nanocell composition comprises a nanocore with at least one first therapeutic consisting of an antifribrotic agent such as colchine and a nanoshell with at least one second therapeutic consisting of a corticosteroid, such as, for example, cortisol, cortisone, hydrocortisone, fludrocortisone, prednisone, methylprednisonlone, or prednisolone etc.

A method for the treatment of idiopathic pulmonary fibrosis utilizing the tailored nanocell composition is also disclosed. In this method, an individual is administered a tailored nanocell of the present invention via inhalation.

A method for synthesizing the tailored compositions of the present invention is also disclosed.

Kits with the necessary agents needed to assemble the novel nanocells and practice the methods of the present invention are also provided.

### BRIEF DESCRIPTION OF THE DRAWINGS

FIG. 1 shows a model of the nuclear nanocell of the present invention. The radionuclide containing nanocore is surrounded by a lipid nanoshell which is modified with PEG.

FIG. 2 shows localization of nanocells in vivo. Tumor cells were implanted in mice and allowed to grow into solid tumors. The animals were injected with the modified nanocells and were sacrificed at 10 and 24 hours post-administration. The tissues were fixed and stained for blood vessels. The results show the blood vessel, modified nanocell, and a merge of the two in spleen, liver, and lungs. As shown in these confocal images, there is limited uptake into the spleen and the nanocells are only present in the blood vessels and tumor.

In FIG. 3, tumor cells were implanted in mice and allowed to grow into solid tumors. The animals were injected with nanocells with a quantum dot core, and sacrificed at 10 h and 24 h post-administration. The tissues were harvested, fixed, and stained for blood vessels. The images shown are depth coding, showing the distribution of the nanocells in a 3-dimension by merging images on the z-axis. As shown in the confocal images, there is limited uptake into the spleen, and is restricted in the vasculature of lungs and liver, but extravasates out in the tumor.

FIG. 4 shows a model of a generic nanocell without tailoring to treat a particular disease.

FIGS. 5A-5D: FIGS. 5A and 5B show electron micrographs of a nanocell tailored for treatment of asthma. FIG. 5C shows that a bronchodilator, salbutamol, is released rapidly, while FIG. 5D shows that a corticosteroid, Dexamethasome, is released over hours.

FIG. 6 shows the effect of nanocell treatment on inflammation associated with asthma. Following the administration of nanocells (comprised of salbutamol and dexamethasone), the inflammation, as quantified by measuring infiltrated cells in lungs, is significantly lower as compared with a equivalent dose of a regular combination. This indicates that the present nanocells result in improved efficacy.

FIG. 7 shows the sequence of a TF antigen-binding peptide (SEQ ID NO:1).

FIG. 8 shows a synthetic scheme for the generation of Tf-antigen-selective quantum dot conjugate.

FIG. 9 shows FRET between quantum dot 565 and fluorescently-labeled asialofetuin. The quantum dot is excited at 450 nm and emits at 565 nm. In the presence of FRET acceptor (fluorescently-labeled asialofetuin) the 565 nm emission band of the nancrsytal is quenched via FRET by the alexa fluor 610 on the asialofetuin. After saturation with excess asialofetuin (37 nM) free TF antigen was added (3.9 and 7.8 μM) was added resulting in recovery of 565 nm fluorescence (arrows in the graph) indicating the dissociation of the nanocrystal and the TF antigen.

FIG. 10 shows the selective targeting of malignant tissue using the quantum dot conjugate.

FIG. 11A through 11I: FIG. 11 shows selectivity of the conjugate for different malignant tissue: (11A) Brain tumor, (11B) Lung cancer, (11C) breast cancer, (11D) melanoma, (11E) head and neck cancer, (11F) Colon cancer, (11G) ovarian cancer (11H) non hodgkin's lymphoma, (11I) prostate cancer.

FIG. 12 shows C57/BL6 mice injected with B16/F10 melanoma cells. Q-Dots labeled with random hexamer sequence and the TF antigen-binding peptide are imaged in green while the vasculature is imaged in red.

### DETAILED DESCRIPTION OF THE INVENTION

Imaging Compositions and Methods for Detecting Disease or Disorder

We have now discovered compositions and methods for readily delivering imaging agents and radionuclides to a desired target. The compositions and methods take advantage of nanocells. One can bind the radionuclide to the nanocell by a variety of means as discussed below. Using the methods of the invention, one can complex the quantum dot or a imaging agent or a radionuclide to the nanocell with a ligand without the need to make sure that this ligand also targets the desired tissue to be imaged. For example, one can use a ligand that readily complexes with a radionuclide such as Tc-99m to bind to the nanocell without regard to what target this ligand will bind to because the radionuclide-nanocell complex will target the desired tissue, not the ligand-radionuclide complex. The ligand-radionuclide complex is used to bind the radionuclide to the nanocell.

In another embodiment, the nanocell comprises a light emitting quantum dot or fluorescent-nanocore nucleated in a lipid matrix or nanoshell. The lipid nanoshell could be pegylated and ligands or peptides for targeting to specific tissues can be linked to the lipids or the PEG.

This can be done by a number of means. For example, one can use nanocells of specified sizes and/or size ranges to deliver the imaging nanocells to certain targets. Most tumors have larger pores (400-600 nm) in their vasculature than normal cells. Therefore, by using radionuclide-nanocells, such as Tc-99m nanocells, that have a size range larger than the pores on normal cells, e.g. preferably at least 55 nm, more preferably at least 60 nm, one can target malignant organs, tissues and cells. A preferred size range is 60-600 nm. Other ranges can be about 75-250 nm. However, one can use any size range from 60-600 nm, e.g. 60, 65, 70, 75, 80, 85, 90, 95, 100, up to 600 nm.

In another embodiment, the radionuclide-nanocells is targeted to specific tissues by using a ligand on the nanocell that targets specific cells. In a preferred embodiment, the ligand is attached to the nanocell on its lipid nanoshell or PEG. In such an embodiment, the nanocell size range is 5-50 nm, preferably 30-45 nm.

These imaging compositions can be used in a wide range of applications. For example, screening for changes in uptake in specific tissues, for diagnosis and for prognosis. In one embodiment one can look at angiogenic diseases and disorders, e.g. tumors, in vivo. Other angiogenic diseases where this would be used are arthritis, tissue regeneration, diabetic retinopathy, etc.

More specifically, nanoparticles, such as nanocells (see U.S. Patent Application No. 60/549,280, filed Mar. 2, 2004) are modified to contain a radioactive nanocore that can be readily imaged. In one embodiment, the radionuclide is chemically linked or adsorbed to a polymer, preferably a biodegradable polymer. One preferred radionuclide is Tc-99m. However, any radionuclide can be used. In one preferred embodiment, the radionuclides are size restricted to greater than about 60 nm so that they selectively extravasate at sites of angiogenesis (e.g. tumor) and do not pass through normal vasculature or enter non-tumor bearing tissue. The radionuclide containing nanocell comprises an inner nanocore of radionuclide, an outer nanoshell of lipid with associated PEG. Thus, in one embodiment, the present invention describes novel radioimaging agents and methods for their use in solid tumor detection or in treatment.

In a preferred embodiment of the present invention, the nuclear nanocell is about 60 nm to about 120 nm in total diameter. Preferably, the size will be between about 60 nm and about 120 nm, more preferably between about 60 nm and about 80 nm or between about 60 nm to about 90 nm. Alternatively, the modified radioactive nanocell may be from about 60 nm to about 600 nm in total diameter.

Composition of Imaging Nanocell

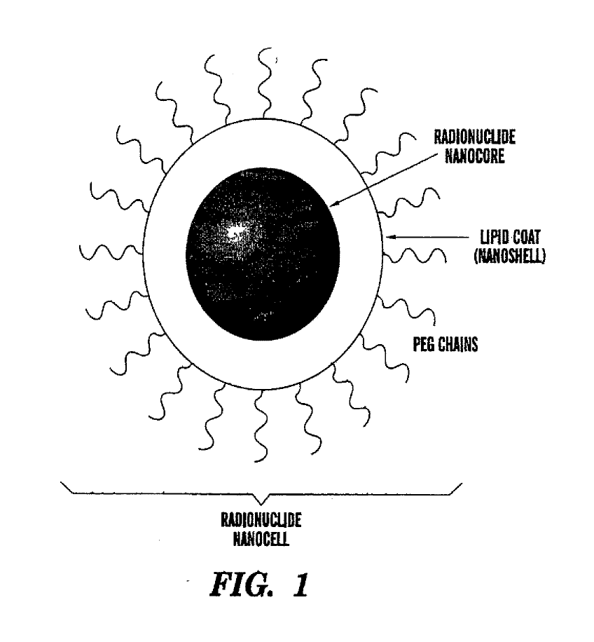

The radioactive nanocell of the present invention comprises 1) an inner nanoparticle (also known as the nanocore) that contains an imaging agent, preferably a radionuclide; 2) an outer nanoshell comprised of lipid; and 3) polyethylene glycol (PEG). An example is shown in FIG. 1.

The nanocell may further comprise targeting moieties or ligands that specifically target the nanocell to specific organs, tissue or cells. Such a targeting ligands may be attached to the outer surface of the nanocell (i.e. on the PEG or lipid nanoshell) to further enhance and target delivery of the nanocell.

Proteins with desired binding characteristics such as specific binding to another protein (e.g. receptors), binding to ligands (e.g. cAMP, signaling molecules) and binding to nucleic acids (e.g. sequence-specific binding to DNA and/or RNA), binding to sugars may be utilized. Haptens, enzymes, antibodies, antibody fragments, cytokines, receptors, hormones, and other small proteins, polypeptides, or non-protein molecules which confer particular surface recognition feature to the nanocells may be utilized. Techniques for coupling surface molecules to lipids are known in the art (see, e.g., U.S. Pat. No. 4,762,915).

For example, the nanocells can be tailored so as to target cancer-associated carbohydrates in different tissues. The carbohydrate pattern of malignant cells differs from that of normal cells. Thus, one can use a ligand or antibody directed to the different carbohydrate to selectively bind to the desired cell. In one embodiment, nano-scale scaffolds are utilized to display carbohydrate-binding molecules in multivalent fashion in order to increase the selectivity and affinity of the conjugates to the cancer-associated carbohydrate. These scaffolds may be conjugated to different imaging probes. This can be used to image the selectivity of the conjugates for malignant tissue or treat the malignant cells. In one embodiment, synthetic peptides are displayed on the nanocell in a multivalent fashion so as to selectively target cancer-associated carbohydrates on the surface of cancer cells. For example, many cancer-associated mucins show increases in core type 1, Thomsen-Fridenreich antigent (TF antigen), and immunodominant Galβ1-3Gal-NA_{cu} disaacharide that is found sialylated on normal cells but nonsialylated in carcinoma cells. The TF antigen-binding peptide is utilized and is modified to incorporate a thiol functional group at the N-terminus for selective conjugation to maleimides inserted at the end of the polyethylene glycol (PEG) spacers on the surface of the nanocells (e.g. on the nanoshell). The PEG spacers between the quantum dot and the peptide increase the flexibility of the peptide and therefore facilitate the multivalent interaction with their antigen on cell surfaces. In another embodiment, the ligands may be incorporated into the nanocore.

In one embodiment, synthetic peptides are incorporated into the nanocell for targeting desired tissues. The peptides, for example, SEQ ID NO. 1 (1-V-W-H-R-W-Y-A-W-S-P-A-S-R-I) or PrPUP may be synthesized as is known to those of skill in the art, for example, on PAL-PEG-PS resin by using an automated ACT peptide synthesizer. The peptides may be prepared as the C-terminal amide and the N-terminal acetyl derivative. Standard 9-fluorenylmethoxycarbonyl (Fmoc) chemistry and HBTU/HOBT activation may be used for all residues except cysteine. Preactivated Fmoc-L-Cys(Trt)-OPfp may be used in the absence of base to prevent racemization.

In particular, nanocells may be modified so that their surfaces contain moieties that directly and efficiently interact with cellular targets both on the cell surface and/or intracellularly. In one embodiment, the targeting moiety may comprise two distinct targeting moieties that independently interact with cellular targets. For example, a first targeting moiety interacts with a first cellular target and a second targeting moiety interacts with a second cellular target, such as an intracellular target. Alternatively, the targeting moiety may comprise two distinct targeting moieties that dependently interact with cellular targets. For example, the first and second targeting moiety target one cellular target. In another embodiment of the present invention, the nanocell comprises a targeting moiety that specifically interacts with a homo- or hetero-dimerized or trimerized cellular receptor. In this embodiment, the targeting moiety is specific for the dimerized or trimerized cellular receptor and, for example, does not interact with another form such as the non-dimerized or trimerized form.

One can also control the number of targeting moieties on a particular particle. For example, in one embodiment the particle would contain 1-50 targeting moieties and any combination in between. One can tailor the particle to contain a sufficient number of the targeting moieties to form a desired multimeric complex. Preferably 6-12 targeting moieties.

Suitable targeting moieties may be identified by methods known to those of skill in the art, for example, by testing for selective binding to a cellular receptor and the result of this binding such as activation and or inhibition. Receptor binding may be assayed, for example, by displacement/competitive binding assays using cells expressing the cognate receptors (See generally Ilag et al J. Biol. Chem. 269:19941-19946 and references therein; Ruden et al J. Biol. Chem. 217:5623-5627).

It is understood that the targeting moieties and methods described above may be utilized for targeting nanocells to be used in detecting disease and/or disorder and also in treatment of disease and/or disorder.

In a further embodiment, the nanocell can contain a therapeutic or a caged therapeutic so that in addition to providing diagnostic imaging, the nanocell may also be used as a therapeutic. For example, the invention can also be practiced by including with the diagnostic nanocell of the invention an anti-cancer chemotherapeutic agent such as any conventional chemotherapeutic agent or a therapeutic radionuclide such as rhenium. Numerous chemotherapeutic protocols will present themselves in the mind of the skilled practitioner as being capable of incorporation into the composition of the invention. Any chemotherapeutic agent can be used, including alkylating agents, antimetabolites, hormones and antagonists, radioisotopes, as well as natural products. For example, the nanocell of the invention can be administered with antibiotics such as doxorubicin and other anthracycline analogs, nitrogen mustards such as cyclophosphamide, pyrimidine analogs such as 5-fluorouracil, cisplatin, hydroxyurea, paclitaxel (Taxol™) and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Furthermore, the combined imaging-therapeutic nanocell compositions of the present invention may be tailored for particular release kinetics as described more fully below. For example, the therapeutic may be formulated for slow or fast release depending on the disease or disorder to be diagnosed, detected and treated.

Methods for incorporating therapeutics into the diagnostic nanocell of the present invention are well known to those of skill in the art and are described in detail below. For example, methods for incorporating therapeutics into nanocells or lipid bilayers may be found in U.S. Patent Application No. 60/549,280, filed Mar. 2, 2004 and U.S. Patent Application Publication No. 2005/0025819, published Feb. 3, 2005.

### Preparation of Nanoparticles

Preferably one uses a nanocell, but any nanoparticle can be used. This is accomplished by first preparing an inner nanocore or nanoparticle to be conjugated to a radionuclide. This nanocore may be a quantum dot or any other nanoparticle of sufficient size and composition.

The nanocore preferably contains a radionuclide complex bound in a matrix. The matrix is preferably a polymeric matrix that is biodegradable and biocompatible. Polymers useful in preparing the nanocore include synthetic polymers and natural polymers. These nanocores are prepared using any of the materials such as lipids, proteins, carbohydrates, simple conjugates and polymers (e.g. PLGA, polyesters, polyamides, polycarbonates, poly(beta-amino esters), polycarbamides, polysaccharides, polyaryls, polyureas, polycarbamates, proteins, etc.) and methods (e.g., double emulsion, spray drying, phase inversion, etc.) known in the art. Diagnostic agents can be loaded in the nanocore, or covalently linked, or bound through electrostatic charges, or electrovalently conjugated, or conjugated through a linker.

In relation to the radioactive nanocells of this invention, a “nanometer particle” or “nanoparticle” or “nanocore” refers to a metal or semiconductor particle or a nanoparticle synthesized from a biodegradable polymer with a diameter in the nanometer (nm) range. The polymers useful in the nanocores have average molecular weights ranging from 100 g/mol to 100,000 g/mol, preferably 500 g/mol to 80,000 g/mol. In a preferred embodiment, the polymer is a polyester synthesized from monomers selected from the group consisting of D, L-lactide, D-lactide, L-lactide, D, L-lactic acid, D-lactic acid, L-lactic acid, glycolide, glycolic acid, epsilon-caprolactone, epsilon-hydroxy hexanoic acid, gamma-butyrolactone, gamma-hydroxy butyric acid, delta-valerolactone, delta-hydroxy valeric acid, hydroxybutyric acids, and malic acid. More preferably, the biodegradable polyester is synthesized from monomers selected from the group consisting of D, L-lactide, D-lactide, L-lactide, D, L-lactic acid, D-lactic acid, L-lactic acid, glycolide, glycolic acid, epsilon-caprolactone, and epsilon-hydroxy hexanoic acid. Most preferably, the biodegradable polyester is synthesized from monomers selected from the group consisting of D, L-lactide, D-lactide, L-lactide, D, L-lactic acid, D-lactic acid, L-lactic acid, glycolide, and glycolic acid. Copolymers may also be used in the nanocore. Copolymers include ABA-type triblock copolymers, BAB-type triblock copolymers, and AB-type diblock copolymers. The block copolymers may have hydrophobic A blocks (e.g., polyesters) and hydrophilic B block (e.g., polyethylene glycol).

The nanoparticles may be any size that can be encapsulated in a lipid nanoshell having a minimum diameter of approximately 5 nm and a maximum diameter of approximately 600 nm.

The metal can be any metal, metal oxide, or mixtures thereof. Some examples of metals useful in the present invention include gold, silver, platinum, and copper. Examples of metal oxides include iron oxide, titanium oxide, chromium oxide, cobalt oxide, zinc oxide, copper oxide, manganese oxide, and nickel oxide.

The metal or metal oxide can be magnetic. Examples of magnetic metals include, but are not limited to, iron, cobalt, nickel, manganese, and mixtures thereof. An example of a magnetic mixture of metals is a mixture of iron and platinum. Examples of magnetic metal oxides include, for example, iron oxide (e.g., magnetite, hematite) and ferrites (e.g., manganese ferrite, nickel ferrite, or manganese-zinc ferrite).

Preferably, the nanoparticle comprises a semiconductor. Some examples of semiconductors include Group II-VI, Group III-V, and Group IV semiconductors. The Group II-VI semiconductors include, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, and mixtures thereof. Group III-V semiconductors include, for example, GaAs, GaN, GaP, GaSb, InGaAs, InP, InN, InSb, InAs, AlAs, AlP, AlSb, AlS, and mixtures therefore. Group IV semiconductors include, for example, germanium, lead, and silicon.

The semiconductor may also include mixtures of semiconductors from more than one group, including any of the groups mentioned above.

The formation of nanoparticles comprising Group III-V semiconductors is described in U.S. Pat. No. 5,751,018 and U.S. Pat. No. 5,505,928. U.S. Pat. No. 5,262,357 describes Group II-VI and Group III-V semiconductor nanoparticles. These patents also describe the control of the size of the semiconductor nanoparticles during formation using crystal growth terminators. The specifications of U.S. Pat. No. 5,751,018, U.S. Pat. No. 5,505,928, and U.S. Pat. No. 5,262,357 are hereby incorporated by reference.

Many semiconductors that are constructed of elements from groups II-VI, III-V and IV of the periodic table have been prepared as quantum sized particles, exhibit quantum confinement effects in their physical properties, and can be used in the composition of the invention. Exemplary materials suitable for use as quantum dots include ZnS, ZnSe, ZnTe, CdS, CdSe, CdTe, GaN, GaP, GaAs, GaSb, InP, InAs, InSb, AlS, AlP, AlAs, AlSb, PbS, PbSe, Ge, and Si and ternary and quaternary mixtures thereof. The quantum dots may further include an overcoating layer of a semiconductor having a greater band gap. The semiconductor nanocrystals are characterized by their uniform nanometer size. Such particles are commercially available and may be utilized in the composition and methods of the present invention.

In one embodiment, the nanoparticles are used in a core/shell configuration. A first semiconductor nanoparticle forms a core ranging in diameter, for example, from about 2 nm to about 10 nm. A shell, of another semiconductor nanoparticle material, grows over the core nanoparticle to a thickness of, for example, 1-10 monolayers. When, for example, a 1-10 monolayer thick shell of CdS is epitaxially grown over a core of CdSe, there is a dramatic increase in the room temperature photoluminescence quantum yield.

The core of a nanoparticle in a core/shell configuration can comprise, for example, MgS, MgSe, MgTe, CaS, CaSe, CaTe, SrS, SrSe, SrTe, BaS, BaTe, BaTe, ZaS, ZnSe, ZnTe, CdS, CdSe, CdTe, HgS, HgSe, HgTe, GaAs, GaN, GaP, GaSb, InGaAs, InP, InN, InSb, InAs, AlAs, AlP, AlSb, AlS, PbS, PbSe, Ge, Si, or mixtures thereof. Examples of semiconductors useful for the shell of the nanoparticle include, ZnO, ZnS, ZnSe, ZnTe, CdO, CdS, CdSe, CdTe, MgS, MgSe, GaAs, GaN, GaP, GaAs, GaSb, HgO, HgS, HgSe, HgTe, InAs, InN, InP, InSb, AlAs, AlN, AlP, AlSb, or mixtures thereof. Preferably, the core/shell comprises CdSe/CdS, CdSe/ZnS, or CdTe/ZnS. Formation of such core/shell nanoparticles is described more fully in Peng et al., Epitaxial Growth of Highly Luminescent CdSe/CdS Core/Shell Nanoparticles with Photostability and Electronic Accessibility, Journal of the American Chemical Society, (1997) 119:7019-7029, the subject matter of which is hereby incorporated by reference.

In a preferred embodiment of the present invention, the nanocore is water soluble. Quantum dots described by Bawendi et al. (J. Am. Chem. Soc., 115:8706, 1993) are soluble or dispersible only in organic solvents, such as hexane or pyridine.

The nanocore may be prepared using any method known in the art for preparing nanoparticles. Such methods include spray drying, emulsion-solvent evaporation, double emulsion, and phase inversion. In addition, any nanoscale particle, matrix, or core may be used as the nanocore inside the nanocell. The nanocore may be, but is not limited to, nanoshells (see U.S. Pat. No. 5,858,862), nanocrystals (see U.S. Pat. No. 6,114,038), quantum dots (see U.S. Pat. No. 6,326,144), and nanotubes (see U.S. Pat. No. 6,528,020).

A critical feature of the present invention is the size of the nuclear nanocell. Thus, the radionuclide nanocore is size restricted so that the total diameter of the nanocell is no smaller than 60 nm. Methods to size restrict nanoparticles is known in the art. In general, once prepared (with or without radionuclide), the nanocores may be fractionated by filtering, sieving, extrusion, or ultracentrifugation to recover nanocores within a specific size range. One effective sizing method involves extruding an aqueous suspension of the nanocores through a series of polycarbonate membranes having a selected uniform pore size; the pore size of the membrane will correspond roughly with the largest size of nanocores produced by extrusion through that membrane. See, e.g., U.S. Pat. No. 4,737,323, incorporated herein by reference. Another preferred method is ultracentrifugation at defined speeds to isolate fractions of defined sizes.

Nanoparticle Plus Radionuclide

The radionuclide is combined with the quantum dot or nanoparticle to create the nanocore. In a preferred embodiment, technetium-99m (^{99m}Tc or 99m-Tc) is used due to its excellent physical decay properties and its chemistry. Other radionuclides for imaging are known and may be used. Typical diagnostic radionuclides include, (95)Tc, (111)In, (62)Cu, (64) Cu, (67)Ga, (48)F and (68)Ga.

For diagnostic purposes Tc-99m is the preferred isotope. Its 6 hour half-life and 140 keV gamma ray emission energy are ideal for gamma scintigraphy using equipment and procedures well established for those skilled in the art. The rhenium isotopes also have gamma ray emission energies that are compatible with gamma scintigraphy, however, they also emit high energy beta particles that are more damaging to living tissues. However, these beta particle emissions can be utilized for therapeutic purposes, for example, cancer radiotherapy, and thus may be utilized in the composition and methods of the present invention for combination diagnostic and therapeutic purposes.

Exemplary procedures for conjugating technetium to ligands are disclosed, for example, in U.S. Pat. No. 4,826,961, European Patent Application 1293214, Cerqueira et al., Circulation, Vol. 85, No. 1, pp. 298-304 (1992), Pak et al., J. Nucl. Med., Vol. 30, No. 5, p. 793, 36th Ann. Meet. Soc. Nucl. Med. (1989), Epps et al., J. Nucl. Med., Vol. 30, No. 5, p. 794, 36th Ann. Meet. Soc. Nucl. Med. (1989), Pak et al., J. Nucl. Med., Vol. 30, No. 5, p. 794, 36th Ann. Meet. Soc. Nucl. Med. (1989), and Dean et al., J. Nucl. Med., Vol. 30, No. 5, p. 79.sup.4, 36th Ann. Meet. Soc. Nucl. Med. (1989), the disclosures of each of which are hereby incorporated herein by reference, in their entirety.

The technetium radionuclides are preferably in the chemical form of pertechnetate or perrhenate and a pharmaceutically acceptable cation. The pertechnetate salt form is preferably sodium pertechnetate such as obtained from commercial Tc-99m generators. The amount of pertechnetate used to prepare the radiopharmaceuticals of the present invention can range from 0.1 mCi to 1 Ci, or more preferably from 1 to 200 mCi.

The radionuclide can be provided to a preformed emulsion of nanocores in a variety of ways. For example, (99)Tc-pertechnate may be mixed with an excess of stannous chloride and incorporated into the preformed emulsion of nanocells. Stannous oxinate can be substituted for stannous chloride. Means to attach various radionuclides to the nanocells of the invention are understood in the art.

Generally, radionuclide nanocores are prepared by procedures which introduce the radionuclide at a late stage of the synthesis. This allows for maximum radiochemical yields, and reduces the handling time of radioactive materials. When dealing with short half-life isotopes, a major consideration is the time required to conduct synthetic procedures, and purification methods. Protocols for the synthesis of radiopharmaceuticals are described in Tubis and Wolf, Eds., “Radiopharmacy”, Wiley-Interscience, New York (1976); Wolf, Christman, Fowler, Lambrecht, “Synthesis of Radiopharmaceuticals and Labeled Compounds Using Short-Lived Isotopes”, in Radiopharmaceuticals and Labeled Compounds, Vol 1, p. 345-381 (1973), the disclosures of each of which are hereby incorporated herein by reference, in their entirety.

Radionuclides such as rhenium-186m and particularly, technetium-99m, are typically conjugated to ligands to form a radionuclide complex, and in particular peptide ligands, via relatively stable bonds with a sulfhydryl group. However, for suithydryl group-bonding to occur, rhenium-186m and technetium-99m must be in the +3, +4 or +5 oxidation state, Because technetium-99m is most readily available as its pertechnetate-99m salt, i.e., a form of technetium having a +7 oxidation state, most technetium-99m species must be reduced prior to reaction with a sulfhydryl group.

The labeling of biomolecule sulfhydryl groups via reduction of pertechnetate-99m salt has been performed using stannous (Sn²⁺) ion as a reducing agent for technetium-99m. In particular, aqueous solutions of stannous ion formed from acidic solutions (D. W. Wong et al., Int. J. appl. Radiat. Isotopes, 29, 251 (1978); A. Schwarz et al., Abstract No. 695 from the “Proceedings of the 34th Annual Meeting,” J. Nucl. Med., Vol. 28, No. 4, April 1987; B. A. Rhodes, Nucl. Med. Biol., 18(7), 667 (1991); G. L. Griffiths et al., Bioconjugate Chem., 3(2), 91 (1992); EP Patent Application 403 225 to Immunomedics, Inc.; U.S. Pat. No. 4,305,992 to Rhodes and U.S. Pat. No. 5,334,708 to Chang et al.); stannous ion in the presence of tartrate anion (B. A. Rhodes et al., J. Nucl. Med., 27(5), 685 (1986); G. L. Griffiths et al., Nucl. Med. Biol., 21(4), 649 (1994); U.S. Pat. No. 5,061,641 to Shocat et al.; U.S. Pat. No. 4,877,868 to Reno et al.; U.S. Pat. Nos. 5,346,687, 5,277,893, 5,102,990 and 5,078,985 to Rhodes; U.S. Pat. Nos. 4,424,200 and 4,323,546 to Crockford et al.; U.S. Pat. Nos. 4,472,371 and 4,311,688 to Burchiel et al.; U.S. Pat. No. 5,328,679 to Hansen et al.; and EP Patent Applications 419 203 and 336 678 to Immunomedics, Inc.); stannous ion in the presence of glucarate (K. Y. Pak et al., Abstract No. 268 from the “Proceedings of the 36th Annual Meeting,” J. Nucl. Med., Vol. 30, No. 793 (1989); K. Y. Pak et al., J. Nucl. Med., 33, 144 (1992); A. F. Verbruggen, Eur. J. Nucl. Med., 17, 346 (1990)); stannous ion in the presence of benzoic acid derivatives (S. J. Mather et al., J. Nucl. Med., 31, 692 (1990); U.S. Pat. No. 4,666,698 to Schwarz; PCT Publication No. 85/03231 to Institutt for Energiteknikk; and U.S. Pat. No. 5,164,175 to Bremer); stannous ion in the presence of diethylenetriaminepentaacetic acid derivatives (U.S. Pat. Nos. 4,668,503 and 4,479,930 to Hnatowich; U.S. Pat. No. 4,652,440 to Paik et al.; and U.S. Pat. No. 4,421,735 to Haber et al.); stannous ion in the presence of saccharic acid (U.S. Pat. No. 5,317,091 to Subramanian; WO 88/07382 to Centocor Cardiovascular Imaging Partners, L.P.;) stannous ion in the presence of glucoheptonate (U.S. Pat. No. 4,670,545 to Fritzberg et al.); stannous ion in the presence of D-gluconate (U.S. Pat. No. 5,225,180 to Dean et al.) have been used to effect technetium-99m labeling of sulfhydryl group-bearing peptides. In addition, dithionite has been used as the reducing agent for pertechnetate-99m (U.S. Pat. No. 4,647,445 to Lees).

The labeling of sulfhydryl group-bearing peptides using 99m TcNCl(4) has also been described (WO 87/04164 to the University of Melbourne).

Preferably, the radionuclide is ligated to a biomolecule in the absence of acids and bases following the methods of U.S. Pat. No. 6,080,384. In general, this method provides for labeling sulfhydryl group-bearing biomolecules with a radionuclide, wherein a stannous salt used to reduce the radionuclide is premixed with a water-miscible organic solvent. The radionuclide can be rhenium-186m, preferably in the form of perrhenate-86m salt, or the radionuclide can be technetium-99m, preferably in the form of pertechnetate-99m salt. In a preferred embodiment of the invention, the radionuclide is technetium-99m, in the form of a pertechnetate-99m salt.

Alternatively, the radionuclide may be indirectly conjugated using a chelating agent. Candidates for use as chelators are those compounds that bind tightly to the chosen metal radionuclide and also have a reactive functional group for conjugation with the targeting molecule. For utility in diagnostic imaging, the chelator desirably has characteristics appropriate for its in vivo use, such as blood and renal clearance and extravascular diffusibility.

For diagnostic imaging purposes, the chelators are used in combination with a metal radionuclide. Suitable radionuclides include technetium and rhenium in their various forms such as 99m TcO(3−), 99m TcO(2+), ReO(3+) and ReO(2+).

Chelation of the selected radionuclide can be achieved by various methods. Typically, a chelator solution is formed initially by dissolving the chelator in aqueous alcohol e.g. ethanol-water 1:1. The solution is degassed with nitrogen to remove oxygen then sodium hydroxide is added to remove the thiol protecting group. The solution is further purged with nitrogen and heated (e.g. on a water bath) to hydrolyse the thiol protecting group, and the solution is then neutralized with an organic acid such as acetic acid (pH 6.0-6.5). In the labeling step, sodium pertechnetate is added to the chelator solution with an amount of stannous chloride sufficient to reduce the technetium. The solution is mixed and left to react at room temperature and then heated (e.g. on a water bath). In an alternative method, labeling can be accomplished as with the chelator solution adjusted to pH 8. Pertechnetate may be replaced with a solution of technetium complexed with labile ligands suitable for ligand exchange reactions with the desired chelator. Suitable ligands include tartarate, citrate or heptagluconate. Stannous chloride may be replaced with sodium dithionite as the reducing agent if the chelating solution is alternatively adjusted to pH 12-13 for the labeling step. The labeled chelator may be separated from contaminants 99m TcO₄ and colloidal 99m TcO₂ chromatographically, e.g., with a C-18 Sep Pak column activated with ethanol followed by dilute HCl. Eluting with dilute HCl separates the 99m TcO₄, and eluting with EtOH-saline 1:1 brings off the chelator while colloidal 99m TcO₂ remains on the column.

In general, a radionuclide coordination complex of an isonitrile ligand and a radioactive metal selected from the class consisting of radioactive isotopes of Tc, Ru, Co, Pt, Fe, Os, Ir, W, Re, Cr, Mo, Mn, Ni, Rh, Pd, Nb and Ta, is formed by admixing said ligand with a salt of a displaceable metal having a complete d-electron shell selected from the class consisting of Zn, Ga, Cd, In, Sn, Hg, Tl, Pb and Bi to form a soluble metal-isonitrile salt, and admixing said metal-isonitrile salt with said radioactive metal in a suitable solvent to displace the displaceable metal with the radioactive metal.

This radionuclide-ligand complex is then added to the nanoparticle or QD to form the nanocore immediately prior to use so as to maximize radiochemical yields.

In another embodiment of this invention, a method is provided for preparing radioimaging-nanoparticle complexes (i.e. nanocores) that are substantially free of the reaction materials used to produce the radioimaging complex. The method comprises forming the radioimaging complex by admixing in a suitable solvent in a container a target-seeking ligand or salt or metal adduct thereof, a radionuclide label such as, for instance, technetium-99m, a nanoparticle or QD and a reducing agent, if required, to form the radioimaging complex; coating the interior walls of the container with the radioimaging complex; discarding the solvent containing non-complexed ligand and radionuclide, non-used starting reaction materials and oxidized reducing agent if present; and dissolving the desired radioimaging complex from the container walls with another solvent to obtain said complex substantially free of starting reaction materials and unwanted reaction by-products. The method can also include one or more rinsing steps to further remove starting reaction materials and unwanted reaction by-products to obtain said complex essentially free of such starting materials and by-products.

Methods of stabilizing radionuclide-containing compositions are known to those of skill in the art, e.g. U.S. Patent Application No. 2002187099, and may be utilized in the present invention.

Preparation of Nanoshell

In one embodiment, the nanocore in encased in an outer layer (also known as the nanoshell) that comprises lipid or peptides. Various methods of preparing lipid vesicles have been described including U.S. Pat. Nos. 4,235,871; 4,501,728; 4,837,028; U.S. Patent Application Publication No.: 2004/0033345; PCT Application Publication No. WO 96/14057, each incorporated herein by reference. Any lipid including surfactants and emulsifiers known in the art is suitable for use in the nanocells of the present invention. The lipid component may also be a mixture of different lipid molecules. In a preferred embodiment, the lipids are commercially available and include natural as well as synthetic lipids. The lipids may be chemically or biologically altered. Lipids useful in preparing the inventive nanocell include, but are not limited to, phospholipids, phosphoglycerides, phosphatidylcholines, dipalmitoyl phosphatidylcholine (DPPC), dioleyphosphatidyl ethanolamine (DOPE), dioleyloxypropyltriethylammonium (DOTMA), dioleoylphosphatidylcholine, cholesterol, cholesterol ester, diacylglycerol, diacylglycerolsuccinate, diphosphatidyl glycerol (DPPG), hexanedecanol, fatty alcohols such as PEG and others known to those of skill in the art. The lipid may be positively charged, negatively charged, or neutral. In certain embodiments, the lipid is a combination of lipids

The lipid vesicle portion of the nanocell may be multilamellar or unilamellar.

In one embodiment, the nanoshell, or lipid coat, is prepared separately from the nanocore and combined with the radionuclide nanocore prior to use so as to maximize radionuclide yields. Methods to prepare the lipid nanoshell are described in U.S. Patent Application No. 60/549,280, filed Mar. 2, 2004; U.S. Patent Application Publication No. 2005/0025819, filed Sep. 7, 2004; in Dubertret et al., Science Vol 298, 29 Nov. 2002; U.S. Pat. Nos. 4,235,871; 4,501,728; 4,837,028; and PCT Application Publication No. WO 96/14057, incorporated herein by reference. In one preferred embodiment, the nanocore is encapsulated in a phospholipid block copolymer envelope. In one embodiment of the present invention, this block co-polymer envelope is a sterically-stabilised liposome composed of a mixture of 2000-poly-(ethylene glycol) disteraroylphosphatidylethanolamine (PEG-DSPE), phosphatidylcholine, and cholesterol.

Any lipid including surfactants and emulsifiers known in the art are suitable for use in the nanoshell component of the imaging nanocell of the present invention. For example, the lipid component may be a mixture of different lipid molecules, may be extracted and purified from a natural source or may be prepared synthetically in a laboratory.

In a preferred embodiment, the nanocell also contains polyethylene glycol (PEG), which is preferentially surface exposed, e.g. on the outside of the lipid bilayer. The PEG prevents the nanocell from being taken up by the reticuloendothelial system (RES) or by normal tissues.

According to one aspect of the present invention, polyethylene-glycol (PEG) is covalently conjugated to disteraroylphosphatidylethanolamine (DSPE) (or any other lipid used in the preparation of the nanoshell of the present invention). The PEG-DSPE forms micelles with a hydrophobic core consisting of distearoyl phosphatidylethanolamine (DSPE) fatty acid chains which is surrounded by a hydrophilic “shell” formed by the PEG polymer. The presence of the PEG polymer on the lipid coat prevents the nanocell's in vivo detection by the immune system and uptake by the reticuloendothelial system (RES).

The lipid nanoshell of the invention may be produced from combinations of lipid materials well known and routinely utilized in the art to produce micelles and including at least one lipid component covalently bonded to a water-soluble polymer. Lipids may include relatively rigid varieties, such as sphingomyelin, or fluid types, such as phospholipids having unsaturated acyl chains. The lipid materials may be selected by those of skill in the art in order that the circulation time of the micelles be balanced with the optimal in vivo visualization rate.

Lipids useful in coating the nanocores include natural as well as synthetic lipids. The lipids may be chemically or biologically altered. Lipids useful in preparing the inventive nanocells include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol, stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; and phospholipids. The lipid may be positively charged, negatively charged, or neutral. In certain embodiments, the lipid is a combination of lipids. Phospholipids useful in preparing nanocells include negatively charged phosphatidyl inositol, phosphatidyl serine, phosphatidyl glycerol, phosphatic acid, diphosphatidyl glycerol, poly(ethylene glycol)-phosphatidyl ethanolamine, dimyristoylphosphatidyl glycerol, dioleoylphosphatidyl glycerol, dilauryloylphosphatidyl glycerol, dipalmitotylphosphatidyl glycerol, distearyloylphosphatidyl glycerol, dimyristoyl phosphatic acid, dipalmitoyl phosphatic acid, dimyristoyl phosphitadyl serine, dipalmitoyl phosphatidyl serine, phosphatidyl serine, and mixtures thereof. Useful zwitterionic phospholipids include phosphatidyl choline, phosphatidyl ethanolamine, sphingomyeline, lecithin, lysolecithin, lysophatidylethanolamine, cerebrosides, dimyristoylphosphatidyl choline, dipalmitotylphosphatidyl choline, distearyloylphosphatidyl choline, dielaidoylphosphatidyl choline, dioleoylphosphatidyl choline, dilawyloylphosphatidyl choline, 1-myristoyl-2-palmitoyl phosphatidyl choline, 1-palmitoyl-2-myristoyl phosphatidyl choline, 1-palmitoyl-phosphatidyl choline, 1-stearoyl-2-palmitoyl phosphatidyl choline, dimyristoyl phosphatidyl ethanolamine, dipalmitoyl phosphatidyl ethanolamine, brain sphingomyelin, dipalmitoyl sphingomyelin, distearoyl sphingomyelin, and mixtures thereof. Zwitterionic phospholipids constitute any phospholipid with ionizable groups where the net charge is zero. In certain embodiments, the lipid is phosphatidyl choline.

Cholesterol and other sterols may also be incorporated into the lipid outer portion of the nanocell of the present invention in order to alter the physical properties of the lipid vesicle. utable sterols for incorporation in the nanocell include cholesterol, cholesterol derivatives, cholesteryl esters, vitamin D, phytosterols, ergosterol, steroid hormones, and mixtures thereof. Useful cholesterol derivatives include cholesterol-phosphocholine, cholesterolpolyethylene glycol, and cholesterol-SO₄, while the phytosterols may be sitosterol, campesterol, and stigmasterol. Salt forms of organic acid derivatives of sterols, as described in U.S. Pat. No. 4,891,208, which is incorporated herein by reference, may also be used in the inventive nanocells.

The lipid vesicle portion of the nanocells may be multilamellar or unilamellar. In certain embodiments, the nanocore is coated with a multilamellar lipid membrane such as a lipid bilayer. In other embodiments, the nanocore is coated with a unilamellar lipid membrane.

Derivatized lipids may also be used in the nanocells. Addition of derivatized lipids alter the pharmacokinetics of the nanocells. For example, the addition of derivatized lipids with a targeting agen't may allow the nanocells to target a specific cell, tumor, tissue, organ, or organ system. In certain embodiments, the derivatized lipid components of nanocells include a labile lipid-polymer linkage, such as a peptide, amide, ether, ester, or disulfide linkage, which can be cleaved under selective physiological conditions, such as in the presence of peptidase or esterase enzymes or reducing agents. Use of such linkages to couple polymers to phospholipids allows the attainment of high blood levels for several hours after administration, else it may be subject to rapid uptake by the RES system. See, e.g., U.S. Pat. No. 5,356,633, incorporated herein by reference. The pharmacokinetics and/or targeting of the nanocell can also be modified by altering the surface charge resulting from changing the lipid composition and ratio. Thermal or pH release characteristics can be built into nanocell by incorporating thermal sensitive or pH sensitive lipids as a component of the lipid vesicle (e.g., dipalmitoyl-phosphatidylcholine:distearyl phosphatidylcholine (DPPC:DSPC) based mixtures). Use of thermal or pH sensitive lipids allows controlled degradation of the lipid vesicle membrane component of the nanocell.

Polymers of the invention may thus include any compounds known and routinely utilized in the art of sterically stabilized liposome (SSL) technology and technologies which are useful for increasing circulatory half-life for proteins, including for example polyvinyl alcohol, polylactic acid, polyglycolic acid, polyvinylpyrrolidone, polyacrylamide, polyglycerol, polyaxozlines, or synthetic lipids with polymeric headgroups. The most preferred polymer of the invention is PEG at a molecular weight between 1000 and 5000. Preferred lipids for producing micelles according to the invention include distearoyl-phosphatidylethanolamine covalently bonded to PEG (PEG-DSPE) alone or in further combination with phosphatidylcholine (PC), and phosphatidylglycerol (PG) in further combination with cholesterol (Chol) and/or calmodulin.

Methods of the invention for preparation of sterically stabilized micelle products or sterically stabilized crystalline products can be carried using various techniques. In one aspect, micelle components are mixed in an organic solvent and the solvent is removed using either evaporation or lyophilization. Removal of the organic solvent results in a lipid film, or cake, which is subsequently hydrated using an aqueous solution to permit formation of micelles.

In a more simplified preparation technique, one or more lipids are mixed in an aqueous solution after which the lipids spontaneously form micelles. The resulting micelles are mixed with an amphipathic compound which associates with the micelle products and assumes a more favorable biologically active conformation. Preparing micelle products by this method is particularly amenable for large scale and safer preparation and requires a considerable shorter time frame than methods previously described. The procedure is inherently safer in that use of organic solvents is eliminated.

Preparation of Imaging Nanocell

The nanocore, now complexed with radionuclide, is mixed with the lipid-PEG nanoshell to form the radionuclide nanocell of the present invention. Methods of admixing nanoparticles with lipid outer layers is known to those of skill in the art and described in U.S. Patent Application No. 60/549,280, filed Mar. 2, 2004, incorporated herein by reference.

In one embodiment, the lipids are dissolved in a suitable organic solvent or solvent system and dried under vacuum or an inert gas to form a thin lipid film. Optionally, the film may be redissolved in a suitable solvent, such as tertiary butanol, and then lyophilized to form a more homogeneous lipid mixture, which is in a more easily hydrated powder-like form. The resulting film or powder is covered with an aqueous buffered suspension of nanocores and allowed to hydrate over a 15-60 minute period with agitation. The size distribution of the resulting multilamellar vesicles can be shifted toward smaller sized by hydrating the lipids under more vigorous agitation conditions or by adding a solubilizing detergent such as deoxycholate.

In another embodiment, the coating of the nanocore may be prepared by diffusing a lipid-derivatized with a hydrophilic polymer into pre-formed vesicles, such as by exposing pre-formed vesicles to nanocores/micelles composed of lipid-grafted polymers at lipid concentrations corresponding to the final mole percent of derivatized lipid which is desired in the nanocell. The matric, surrounding the nanocore, containing a hydrophilic polymer can also be formed by homogenization, lipid-field hydration, or extrusion techniques.

In another preferred embodiment, vesicle-forming lipids are taken up in a suitable organic solvent or solvent system, and dried or lyophilized in vacuo or under an inert gas to form a lipid film. Any active agents or targeting moieties to be incorporated in the outer chamber of the nanocell, are preferably included in the lipids forming the film. The aqueous medium used in hydrating the dried lipid or lipid/drug is a physiologically compatible medium, preferably a pyrogen-free physiological saline or 5% dextrose in water, as used for parenteral fluid replacement. The nanocores (with radionuclide) are suspended in this aqueous medium in a homogenous manner, and at a desired concentration, prior to the hydration step. The solution can also be mixed with any additional solute components, such as a water-soluble iron chelator, and/or a soluble secondary compound at a desired solute concentration. The lipids are allowed to hydrate under rapid conditions (using agitation) or slow conditions (without agitation). The lipids hydrate to form a suspension of multilamellar vesicles. In general, the size distribution of the vesicles can be shifted toward smaller sizes by hydrating the lipid film more rapidly while shaking. The structure of the resulting membrane bilayer is such that the hydrophobic (non-polar) “tails” of the lipid orient toward the center of the bilayer, while the hydrophilic (polar) “heads” orient towards the aqueous phase.

In another embodiment, dried vesicle-forming lipids, radionuclide-containing nanocores, and any agent(s) (to be loaded in the outer chamber of the nanocell) mixed in the appropriate ratios, are dissolved, with warming if necessary, in a water-miscible organic solvent or mixture of solvents. Examples of such solvents are ethanol, or ethanol and dimethylsulfoxide (DMSO) in varying ratios. The mixture then is added to a sufficient volume of an aqueous receptor phase to cause spontaneous formation of nanocells. The aqueous receptor phase may be warmed if necessary to maintain all lipids in the melted state. The receptor phase may be stirred rapidly or agitated gently. After incubation of several minutes to several hours, the organic solvents are removed, by reduced pressure, dialysis, or diafiltration, leaving a nanocell suspension suitable for human administration.

In one embodiment, the radionuclide-nanocell is formed by adding a radionuclide in an organic solvent to a pre-formed nanocell. In this embodiment, the nanocell minus the radionuclide is pre-prepared by conjugating the nanoparticle to a ligand that will bind a radionuclide and combining with, for example, the lipid-PEG nanoshell. The radionuclide, in an organic solvent, is then added to this pre-prepared nanocell prior to administration to an individual.

In another embodiment, the lipid nanoshell is pre-prepared separately from the nanocore (nanoparticle and ligand) minus the radionuclide. In this embodiment, the radionuclide is mixed with the nanocore and then this radionuclide-nanocore complex is mixed with the nanoshell to form the radionuclide nanocell.

In yet another embodiment, the radionuclide is added to the nanocore (nanoparticle and ligand) and the nanoshell is therein formed on the radionuclide nanocore.

Nanocell Size

An important consideration in the present invention is the total diameter of the nanocell. To be useful as an imaging agent, the nanoparticle must differentially localize to tumors so as to provide a background for imaging. Thus, in one embodiment, directed to imaging tumors, the present invention provides for the nanocell to be size restricted to greater than about 60 nm so that the nanocell extravasates only at sites of angiogenesis, i.e. sites of tumor, and is not taken up in normal tissue. Thus, the total diameter of the nanocell is about 60 nm to about 600 nm; preferentially the total diameter is about 80 nm to about 220 nm.

The nanocell of the present invention is thus fractionated by filtering, sieving, extrusion, or ultracentrifugation to recover nanocells within a specific size range. This size discrimination is typically done before the radionuclide is incorporated into the nanocore. One effective sizing method involves extruding an aqueous suspension of the nanocells through a series of polycarbonate membranes having a selected uniform pore size; the pore size of the membrane will correspond roughly with the largest size of nanocell produced by extrusion through that membrane. See, e.g., U.S. Pat. No. 4,737,323, incorporated herein by reference. Another preferred method is serial ultracentrifugation at defined speeds (e.g., 8,000, 10,000, 12,000, 15,000, 20,000, 22,000, and 25,000 rpm) to isolate fractions of defined sizes.

Radionuclides

As discussed above, diagnostic imaging using radionuclides is well known. Typical diagnostic radionuclides include (99m)Tc, (95)Tc, (111)In, (62)Cu, (64) Cu, (67)Ga, and (68)Ga, Iodine-123, Iodine-131, Ruthenium-97, Copper-67, Cobalt-57, Cobalt-58, Chromium-51, Iron-59, Selenium-75, Thallium-201, and Ytterbium-169.

The radionuclide, technetium-99m, ^{99m}Tc (T_{1/2} 6.9 h, 140 KeV gamma ray photon emission) is a preferred radionuclide for use in imaging because of its excellent physical decay properties and its chemistry. For example, its half-life of about 6 hours provides an excellent compromise between rate of decay and convenient time frame for an imaging study. However, other radionuclides may be used, such as, for example (18)F or (123)I.

Administration

The radionuclide imaging nanocells of the present invention are administered to an individual via methods known to those of skill in the art for administering radionuclide imaging agents. The particular dosage employed need only be high enough to obtain diagnostically useful images, generally in the range of 0.1 to 20 mCi/70 Kg body weight.

Administration of a composition may be by systemic route, including oral, parenteral, sublingual, rectal such as suppository or enteral administration, or by pulmonary absorption. Parenteral administration may be by intravenous injection, subcutaneous injection, intramuscular injection, intra-arterial injection, intrathecal injection, intra peritoneal injection or direct injection or other administration to one or more specific sites.

Access to the gastrointestinal tract, which can also rapidly introduce substances to the blood stream, can be gained using oral enema, or injectable forms of administration. Compositions may be administered as a bolus injection or spray, or administered sequentially over time (episodically) such as every two, four, six or eight hours.

The invention further provides methods of administering the radionuclide nanocell to an individual comprising the steps of: preparing a radionuclide nanocell according to the methods of the invention and administering an effective amount of the radionuclide nanocell to said individual. The nanocell product of the invention may be administered intravenously, intraarterially, intranasally such as by aerosol administration, nebulization, inhalation, or insufflation, intratracheally, intra-articularly, orally, transdermally, subcutaneously. Methods of administration for amphipathic compounds are equally amenable to administration of compounds that are insoluble in aqueous solutions.

In one embodiment, radionuclide-nanocells with a size of about 30 to about 50 nm in total diameter and with targeting ligands are administered to individuals for diagnostic purposes. In this embodiment, the individual is imaged at a time point known to those of skill in the art and dependant on the particular radionuclide used, e.g. after the radionuclide-nanocell has entered all tissues, bound to a target cell, and non-bound nanocells have cleared sufficiently so that there is a target to background differential. This process allows for optimal background to signal ratios and for technetium-99m is at least 2 hours, preferably 6 hours, but may also be 3, 4, 5, 6, 7, 8, 9, 10, 11, 12, or more hours. The time will further vary depending on the radionuclide used.

In an alternative embodiment, a radionuclide-nanocell with a size of about 60 to about 600 nm is administered to an individual for diagnostic purposes. In this embodiment, the individual is imaged at a time point known to those of skill in the art and dependant on the particular radionuclide used, so as to give optimal radionuclide signal. For example, in this embodiment, the individual is imaged after the nanocell has extravasated into any angiogenic areas (e.g. where the vascular pore size is greater than normal vasculature pore size). One preferably uses a radionuclide that will permit imaging after 3 hours, more preferably at 6 or more hours. However, one can also image at periods from 2 hours on, preferably 2-24 hours. The skilled artisan can determine this timing based on the radionuclide used.

Kits

Also encompassed in the present invention are kits for preparing the imaging and tailored therapeutic nanocells of the present invention. Kits in accord with the imaging invention comprise 1) materials necessary for the preparation of the nuclear nanocore and 2) the prepared lipid bilayer-PEG nanoshell. In one embodiment of the invention, the two components are contained in separate, sterile containers and after addition of radionuclide to the nanocore container are admixed.

In one embodiment, the materials necessary for the preparation of the nanocore comprise an adduct of a displaceable metal (as listed above) and an isonitrile ligand and, if required, a quantity of a reducing agent for reducing a preselected radionuclide. Preferably, such kits contain a predetermined quantity of a metal isonitrile adduct and a predetermined quantity of a reducing agent capable of reducing a predetermined quantity of the preselected radionuclide. It is also preferred that the isonitrile ligand and reducing agent be lyophilized, when possible, to facilitate storage stability. If lyophilization is not practical, the kits are stored frozen. The metal-isonitrile adduct and reducing agent are preferably contained in sealed, sterilized containers.

In one embodiment of the invention, a kit for use in making the radionuclide complexes in accord with the present invention from a supply of 99m Tc such as the pertechnetate solution in isotonic saline available in most clinical laboratories includes the desired quantity of a selected isonitrile ligand in the form of a metal-isonitrile adduct to react with a predetermined quantity of pertechnetate, and a predetermined quantity of reducing agent such as, for example, stannous ion in the form of stannous glucoheptanate to reduce the predetermined quantity of pertechnetate to form the desired technetium-isonitrile complex.

Tailored Therapeutic Compositions and Methods for Treating Specific Disease or Disorder

In another embodiment of the present invention, novel nanocell platforms for the treatment of various diseases and disorders are disclosed. In addition, methods for the treatment of specific diseases and disorders utilizing these compositions are disclosed. Nanocells (see U.S. patent application Ser. No. 11/070,731, filed Mar. 2, 2005) can be tailored so that they directly and efficiently deliver appropriate therapies for appropriate lengths of time to relevant biological sites.

In general, the tailored nanocells of the present invention comprise an inner nanocore containing at least one first therapeutic and at least one outer nanoshell comprised of lipid, which contains at least one second therapeutic that differs from the first therapeutic. Alternatively, the nanocore may contain at least one therapeutic that is substantially similar to the at least one therapeutic contained in the nanoshell. In this embodiment, the composition of the matrix encapsulating the first therapeutic differs from the composition of the matrix encapsulating the at least one second therapeutic so that the therapies are released a different times and/or rates. One can also add third, fourth, fifth, or more layers designed to release the same or different agents at specified times.

In one embodiment of the present invention, a novel composition and method for treating a desired angiogenic disease or disorder, e.g. tumors, is disclosed. In this embodiment, the nanocell comprises a nanocore containing a first therapeutic that is selectively chosen so as to act over an extended period of time and a second therapeutic encapsulated within the outer nanoshell that is selectively chosen so as to act immediately and over a shorter period of time. In one preferred embodiment the tailored nanocells are size restricted such as being greater than about 60 nm so that they selectively extravasate at sites of angiogenesis (e.g. tumor, macular degeneration) and do not pass through normal vasculature or enter non-tumor bearing tissue. In a preferred embodiment of the present invention, the tailored nanocell is about 60 nm to about 600 nm in total diameter. The tailored nanocell may also comprise an imaging agent, as described above, for methods combining imaging and treatment.

In one embodiment, the first therapeutic, located in the nanocore, is an anti-neoplastic and the second therapeutic, located in the nanoshell is an anti-angiogenic.

Anti-neoplastic compounds include, but are not limited to, compounds such as Sutent®/SU11248 (sunitinib malate), floxuridine, gemcitabine, cladribine, dacarbazine, melphalan, mercaptopurine, thioguanine, cis-platin, and cytarabine; and anti-viral compounds such as fludarabine, cidofovir, tenofovir, and pentostatin. Further examples of compounds suitable for association with the nanocore include adenocard, adriamycin, allopurinol, alprostadil, amifostine, aminohippurate, argatroban, benztropine, bortezomib, busulfan, calcitriol, carboplatin, daunorubicin, dexamethasone, topotecan, docetaxel, dolasetron, doxorubicin, epirubicin, estradiol, famotidine, foscarnet, flumazenil, fosphenyloin, fulvestrant, hemin, ibutilide fumarate, irinotecan, levocarnitine, idamycin, sumatriptan, granisetron, metaraminol, metaraminol, methohexital, mitoxantrone, morphine, nalbuphine hydrochloride, nesacaine, oxaliplatin, palonosetron, pamidronate, pemetrexed, phytonadione, ranitidine, testosterone, tirofiban, toradol, triostat, valproate, vinorelbine tartrate, visudyne, zemplar, zemuron, and zinecard. Alternatively, the anti-neoplastic may be a radionuclide.

Anti-angiogenic compounds include, but are not limited to anti-VEGF antibodies, including humanized and chimeric antibodies, anti-VEGF aptamers and antisense oligonucleotides, angiostatin, endostatin, interferons, interleukin I, interleukin 12, retinoic acid, and tissue inhibitors of metalloproteinase-1 and -2.

In one embodiment, the tailored nanocell for the treatment of angiogenic diseases and disorders is specific for lung cancer. In this embodiment, the first therapeutic, located in the nanocore, is selected from the group consisting of cisplatin, carboplatin, Iressa, or Gefitinib and the second therapeutic is a corticosteroid. In this embodiment, the nanocell is greater than about 60 nm.

In another embodiment, the tailored nanocell for the treatment of angiogenic diseases and disorders is specific for breast or kidney cancer. In this embodiment, the first therapeutic in doxorubicin and the second therapeutic is a corticosteroid. In this embodiment, the nanocell is greater than about 60 nm.

In another embodiment, the tailored nanocell for the treatment of angiogenic diseases and disorders is specific for skin cancer and/or melanoma. In this embodiment, the first therapeutic in dacarbazine (DTIC) and the second therapeutic is a corticosteroid. In this embodiment, the nanocell is greater than about 60 nm.

In another embodiment, the tailored nanocell for the treatment of angiogenic diseases and disorders is specific for GI tumors. In this embodiment, the first therapeutic is 5-fluorouracil (5-FU) and the second therapeutic is a corticosteroid. In this embodiment, the nanocell is greater than about 60 nm.

As used herein, the term “corticosteroid” refers to any of the adrenal corticosteroid hormones isolated from the adrenal cortex or produced synthetically, and derivatives thereof that are used for treatment of inflammatory diseases, such as arthritis, asthma, psoriasis, inflammatory bowel disease, lupus, and others. Corticosteroids include those that are naturally occurring, synthetic, or semi-synthetic in origin, and are characterized by the presence of a steroid nucleus of four fused rings, e.g., as found in cholesterol, dihydroxycholesterol, stigmasterol, and lanosterol structures. Corticosteroid drugs include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-11β,17-di hydroxy-16α-m-ethyl pregna-1,4-diene-3,20-dione), and highly derivatized steroid drugs such as beconase (beclomethasone dipropionate, which is 9-chloro-11-β,17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone.

Brain Tumor

In one embodiment, a composition and method for the treatment of brain tumors, such as, for example, gliomas, neuronal tumors, anaplastic glioma and meningioma is disclosed. Other brain tumors treatable by the methods and compositions of the present invention include, but are not limited to, astrocytomas, brain stem gliomas, ependymomas, oligodendogliomas, and non-glial originated brain tumors such as medulloblastomas, meningiomas, Schwannomas, craniopharyngiomas, germ cell tumors, pineal region tumors, and secondary brain tumors.

In this embodiment, the nanocell composition comprises a nanocore with at least one first therapeutic consisting of a corticosteroid and a nanoshell with at least one second therapeutic consisting of a chemotherapeutic. As used herein, a chemotherapeutic includes any cancer treatment, such as, chemical agents or drugs, that are selectively destructive to malignant cells and tissues. The corticosteroid may be selected from the group consisting of cortisol, cortisone, hydrocortisone, fludrocortisone, prednisone, methylprednisonlone, prednisolone or the like. Other corticosteroids are known to those of skill in the art and encompassed in the present invention.

The chemotherapeutic, located in the nanoshell may be selected from the group consisting of nitrosurea-based chemotherapy such as, for example, BCNU (carmustine), CCNU (lomustine), PCV (procarbazine, CCNU, vincristine), or temozolomide (Temodar). Other chemotherapeutics are known to those of skill in the art and may be used in the methods of the present invention. They include, for example, alkylating agents, antitumor antibiotics, plant alkaloids, antimetabolites, hormonal agonists and antagonists, and a variety of miscellaneous agents. See Haskell, C. M., ed., (1995) and Dorr, R. T. and Von Hoff, D. D., eds. (1994). The classic alkylating agents are highly reactive compounds that have the ability to substitute alkyl groups for the hydrogen atoms of certain organic compounds. The classic alkylating agents include mechlorethamine, chlorambucil, melphalan, cyclophosphamide, ifosfamide, thiotepa and busulfan. A number of nonclassic alkylating agents also damage DNA and proteins, but through diverse and complex mechanisms, such as methylation or chloroethylation, that differ from the classic alkylators. The nonclassic alkylating agents include dacarbazine, carmustine, lomustine, cisplatin, carboplatin, procarbazine and altretamine.

Clinically useful antitumor drugs include natural products of various strains of the soil fungus Streptomyces, which are also encompassed in the present invention. Drugs of this class include doxorubicin (Adriamycin), daunorubicin, idarubicin, mitoxantrone, bleomycin, dactinomycin, mitomycin C, plicamycin and streptozocin. Plants-based chemotherapies are also encompassed and include the Vinca alkaloids (vincristine and vinblastine), the epipodophyllotoxins (etoposide and teniposide) and paclitaxel (Taxol). In addition, antimetabolites such as methotrexate, 5-fluorouracil (5-FU), floxuridine (FUDR), cytarubine, 6-mercaptopurine (6-MP), 6-thioguanine, deoxycoformycin, fludarabine, 2-chlorodeoxyadenosine, and hydroxyurea are also encompassed in the present invention.

Preferably, the first therapeutic is encapsulated in any biodegradable polymer such as PLGA at defined ratio, so as to provide for sustained or slow-release kinetics of the corticosteroid. The chemotherapeutic is also encapsulated in a biodegradable polymer including PLGA but at a ratio that provides a more immediate but sustained release of a specific agent. The polymer ratio may be tailored empirically so as to adjust treatment to an individual, rather than the current method of same treatment for every individual. For example, Roche's AmpliChip CYP450™, which analyzes an individuals metabolism toward certain drugs may be used to assess the optimal dose required for a particular individual. In this way, a practitioner is able to combine appropriate nanocores (with optimal PHA ratios) with optimal nanoshells to achieve optimal dosing.

Also encompassed in the present invention are methods for the treatment of brain tumors utilizing the tailored nanocell composition of the invention. In this method, an individual is administered a tailored nanocell of the present invention systemically or by directly injecting into the site in need. Preferably, the tumor is resected and the tailored nanocells are delivered to the area of resection at this time.

Therefore, in further aspects of the present invention, the nanocell compositions described herein may be used for the treatment of angiogenic diseases and disorders and malignancy. Within such methods, the nanocell compositions described herein are administered to a patient, typically a warm-blooded animal, preferably a human. A patient may or may not be afflicted with cancer. Accordingly, the above nanocell compositions may be used to prevent the development of a cancer or to treat a patient afflicted with a cancer. Tailored nanocell compositions may be administered either prior to or following surgical removal of primary tumors and/or treatment such as administration of radiotherapy or conventional chemotherapeutic drugs. Administration of the nanocell compositions may be by any suitable method, including administration by intravenous, intraperitoneal, intramuscular, subcutaneous, intranasal, intradermal, anal, vaginal, topical and oral routes.

Asthma

In another embodiment, a composition and method for the treatment of asthma is disclosed. In this embodiment, the nanocell composition comprises a nanocore with at least one first therapeutic consisting of a corticosteroid and a nanoshell with at least one second therapeutic consisting of a bronchodilator. The corticosteroid may be selected from the group consisting of cortisol, cortisone, hydrocortisone, fludrocortisone, fluticasone, prednisone, methylprednisonlone, or prednisolone etc. The bronchodilator may include an anticholinergic, such as ipratropium or a beta-agonist such as albuterol, metaproterenol, pirbuterol, salmeterol, salbutamol or levalbuteral. The nanocell composition for the treatment of asthma allows for an individual to be administered a smaller dose of corticosteroid than is normally attainable due to the administration of the bronchodilator (encased in the nanoshell), which acts first to make available the biological sites of action for the corticosteroid.

Alternatively, anti-IgE may be incorporated into the nanocore of the nanocell alone or in addition to a corticosteroid. Anti-IgE therapy is a long-term therapy and thus should be formulated in the nanocore of the present composition so as to sustain delivery over time. Commercially available anti-IgE includes Xolair™ (omalizumab), which is approved for individuals with moderate to severe persistent asthma, year round allergies and who are taking routine inhaled steroids.

In another embodiment, the tailored-asthma nanocell may comprise Intal™ (cromolyn) and/or Tilade™ (nedocromil), which help prevent asthma symptoms, especially symptoms caused by exercise, cold air and allergies. Cromolyn and nedocromil help prevent swelling in airways. Because cromolyn and nedocromil are preventive, and must be taken on a regular basis to be effective, they are best suited for incorporation into the nanocore of the asthma-tailored nanocell.

In another embodiment, the tailored asthma nanocell contains leukotriene modifiers such as, for example, Accolate™ (zafirlukast), Singulair™ (montelukast), and Zyflo™ (zileuton). Leukotriene modifiers may be incorporated into either the nanocore or nanoshell, but preferably into the nanocore where they act over an extended period of time. Leukotriene modifiers may be incorporated into the nanocell alone or in addition to other therapies.

Although one can use any method to deliver the nanocell, it is preferred that the asthma tailored nanocell is delivered via inhalation.

### Grave's Disease

In another embodiment, a composition and method for the treatment of Grave's Disease is disclosed. In this embodiment, the nanocell composition comprises a nanocore with at least one first therapeutic consisting of iopanoic acid/ipodate sodium and a nanoshell with at least one second therapeutic consisting of an antithyroid drug such as, for example, methimazole, carbimazole, or propylthiouracil. Alternatively, the first therapeutic may be a radioiodine, such as iodine 123. In one embodiment the nanocore comprises radioiodine alone or in combination with iopanoic acid/ipodate sodium. Likewise, the at least one second therapeutic, incorporated in the nanoshell, may be a beta-blocker (i.e. propanolol).

Other beta-blockers useful in the present invention include acebutolol, atenolol, betaxolol, bisoprolol, carteolol, labetalol, metoprolol, nadolol, oxprenolol, penbutolol, pindolol, sotalol, timolol, atenolol,

Preferably, a tailored nanocell of the present invention is delivered systemically via parenteral or enteral routes.

### Cystic Fibrosis

In another embodiment, a composition and method for the treatment of Cystic Fibrosis is disclosed. In this embodiment, the nanocell composition comprises a nanocore with at least one first therapeutic consisting of an antibiotic. In addition to an antibiotic, the core may also contain an optional bronchodilator or steroid. In this embodiment, the nanoshell contains at least one second therapeutic consisting of recombinant human deoxyribonuclease (rhDNase).

Antibiotics are known to those of skill in the art. See, for example, Curr Opin Pulm Med. 2004 November; 10(6):515-23; Ann Pharmacother. 2005 January; 39(1):86-94; Respir Med. 2005 January; 99(1):1-10. Preferred antibiotics include, but are not limited to ciprofloxacin, ofloxacin, tobramycin (including TOBI), gentamicin, azithromycin, ceftazidime, Keflex™ (cephalexin), Ceclor™ (cefaclor), piperacillin and imipenem.

In another embodiment, the tailored cystic fibrosis nanocell comprises 5-nitrosothiol in a form suitable for administration to a CF patient and formulated to maximize contact with epithelial surfaces of the respiratory tract. S-Nitrosoglutathione is the most abundant of several endogenous S-nitrosothiols. It is uniquely stable compared, for example, to S-nitrosocysteine unless specific GSNO catabolic enzymes are upregulated. Such enzymes can include gamma-glutamyl-transpeptidase, glutathione-dependent formaldehyde dehydrogenase, and thioredoxin-thioredoxin reductase. For this reason, co-administration of inhibitors of GSNO prokaryotic or eukaryotic GSNO catabolism may at times be necessary and are encompassed in the present invention. This kind of inhibitor would include, but not be limited to, acivicin given as 0.05 ml/kg of a 1 mM solution to achieve an airway concentration of 1.mu.M S-nitrosoglutathione (GSNO). Preferably, the S-nitrosoglutathione (GSNO) is in concentrations equal to or in excess of 500 nmole/kg (175 mcg/kg). Other nitrosylating agents such as ethyl nitrite may also be used. Thus, the methods and compositions of the present invention comprise a nitrosonium donor including, but not limited to GSNO and other S-nitrosothiols (SNOs) in a pharmaceutically acceptable carrier that allows for administration by nebulized or other aerosol treatment to patients with cystic fibrosis. These compounds may be incorporated into either the nanocore or nanoshell of the cystic fibrosis nanocell of the present invention.

Preferably, an individual is administered a tailored nanocell of the present invention via inhalation.

### Pulmonary Fibrosis

In another embodiment, a composition and method for the treatment of pulmonary fibrosis is disclosed. Pulmonary fibrosis may also be termed Idiopathic Pulmonary Fibrosis, Interstitial Pulmonary Fibrosis, DIP (Desquamative interstitial pneumonitis), UID (Usual interstitial pneumonitis), all of which are encompassed in the present invention. In this embodiment, the nanocell composition comprises a nanocore with at least one first therapeutic consisting of an antifribrotic agent such as colchine (also known as colchicines) and a nanoshell with at least one second therapeutic consisting of a corticosteroid, such as, for example, cortisol, cortisone, hydrocortisone, fludrocortisone, prednisone, methylprednisonlone, or prednisolone etc. The antifibrotic agent may also be selected from the group consisting of Pirfenidone (Deskar; MARNAC, Inc., Dallas, Tex.), colchicine, D-penicillamine, and interferon.

Preferably, an individual is administered a tailored nanocell of the present invention via inhalation.

Some corticosteroids useful for this invention include, but are not limited to, cortisol, cortisone, hydrocortisone fludrocortisone, prednisone, prednisolone, 6-methylprednisolone, triamcinolone, betamethasone, and dexamethasone. However, any of the adrenal corticosteroid hormones isolated from the adrenal cortex or produced synthetically, and derivatives thereof that are used for treatment of inflammation are useful for this invention.

The tailored nanocells of the present invention may contain more than two layers. In one embodiment, the tailored nanocell comprises a plurality of reservoirs where drugs are deposited in layers. Optionally, polymer membranes may be positioned in between the drug-polymer layers for controlled release of various drugs.

In general, the tailored nanocells of the present invention may be administered to individuals as described above, but may also be administered in manner known to those of skill in the art and so as to tailor administration to an individuals needs. For example, dosage may be adjusted appropriately to achieve a desired therapeutic effect. It will be understood that the specific dose level and frequency of dosage for any particular subject may be varied and will depend upon a variety of factors including the activity of the specific therapeutically active agent employed, the metabolic stability and length of action of that agent, the species, age, body weight, general health, dietary status, sex and diet of the subject, the mode and time of administration, rate of excretion, drug combination, and severity of the particular condition. Generally, daily doses of active therapeutically active agents can be determined by one of ordinary skill in the art without undue experimentation, in one or several administrations per day, to yield the desired results.

In the event that the response in a subject is insufficient at a certain dose, even higher doses (or effective higher doses by a different, more localized delivery route) may be employed to the extent that patient tolerance permits. Multiple doses per day are contemplated to achieve appropriate systemic or targeted levels of therapeutic compounds.

### Psoriasis

In another embodiment, a composition and method for the treatment of psoriasis is disclosed. The nanocells may be tailored in such a way that the nanocore would contain an immunosuppressive agent while the shell would contain an anti-angiogenesis or vascular targeting agent. The nanocore would preferably be composed of a biodegradable polymer while the shell shall comprise of lipids.

### Atherosclerosis

In another embodiment, a composition and method for the treatment of atherosclerosis is disclosed. The nanocells may be tailored in a way that the nanocore would contain an chemotherapeutic agent while the nanoshell may contain an anti-angiogenesis or vascular targeting agent. The nanocore would preferably be composed of a biodegradable polymer while the nanoshell is made of lipids.

### Rheumatoid Arthritis

In another embodiment, a composition and method for the treatment of rheumatoid arthritis is disclosed. The nanocells may be tailored in a way that the nanocore would contain an immunosuppressive agent such as a corticosteroid or antibody or a MMP inhibitor while the shell would contain an anti-angiogenesis or vascular targeting agent. The nanocore is preferably composed of a biodegradable polymer while the nanoshell is made of lipids.

The therapeutic tailored nanocells of the present invention are prepared in a similar manner to the methods described above for imaging nanocells. However, where radionuclide is indicated, a therapeutic agent or compound is used. For example, the nanocore preferably contains at least one therapeutic bound in a matrix. The matrix is preferably a polymeric matrix that is biodegradable and biocompatible as described above. The therapeutic tailored nanocells are may be any size, as described more fully above.

The nanocore, now complexed with at least one first therapeutic, is mixed with the lipid-PEG nanoshell, which is also complexed to at least one second therapeutic to form the tailored nanocell of the present invention. Methods of admixing nanoparticles with lipid outer layers is known to those of skill in the art and described in U.S. patent application Ser. No. 11/070,731, filed Mar. 2, 2005, incorporated herein by reference, and described above.

Also encompassed in the present invention are kits for preparing the tailored nanocells of the present invention. Kits in accord with the present invention comprise 1) prepared nanocore with at least one associated first therapeutic and 2) the prepared lipid bilayer-PEG nanoshell with at least one associated second therapeutic. In one embodiment of the invention, the two components are contained in separate, sterile containers and the two are admixed prior to administration. In this way, a nanocell may be tailored to the particular needs of an individual, by, for example, mixing different nanoshells with different nanocores.

In general, the nanocells of the present invention are administered to an individual via methods known to those of skill in the art for administering therapeutic compounds to individuals.

Administration of the nanocell may be via intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The nanocells can be administered parenterally by injection or by gradual infusion over time and can be delivered by peristaltic means.

Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the nanocells of the invention are formulated into conventional oral administration forms such as capsules, tablets and tonics.

For topical administration, the nanocells are formulated into ointments, salves, gels, or creams, as is generally known in the art. The tailored nanocells may also be administered via inhalation.

The nanocells are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual and each disease.

The nanocells useful for practicing the methods of the present invention are of any formulation or drug delivery system containing the active ingredients, which is suitable for the intended use, as are generally known to those of skill in the art. Suitable pharmaceutically acceptable carriers for oral, rectal, topical or parenteral (including inhaled, subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

Access to the gastrointestinal tract, which can also rapidly introduce substances to the blood stream, can be gained using oral enema, or injectable forms of administration. Nanocells may be administered as a bolus injection or spray, or administered sequentially over time (episodically) such as every two, four, six or eight hours.

### DEFINITIONS

Nanocell: According to the present invention, the term “nanocell” refers to a particle in which a nanocore is surrounded or encapsulated in a matrix or shell. In other words, a smaller particle within a larger particle, or a balloon within a balloon. The nanocell has an imaging agent, such as a radionuclide, or a therapeutic agent(s), such as anti-cancer agent, in the nanocore, which is surrounded by a lipid bilayer (i.e. liposome). The lipid bilayer may be modified with PEG. In other embodiments, the nanocore is surrounded by a polymeric matrix or shell.

Nanocore: As used herein, the term “nanocore” refers to any particle within a nanocell. A nanocore may be a microparticle, a nanoparticle, a quantum dot, a nanodevice, a nanotube, or any other composition of the appropriate dimensions to be included within a nanocell. The nanocore comprises an imaging agent, such as a radionuclide, or a therapeutic agent(s), such as anti-cancer agent(s), to be used for visualizing, detection and treatment of angiogenic diseases or disorder, such as, for example, cancer and in particular solid tumors.

As used herein, an “imaging nanocell” may also be termed a “radionuclide nanocell”. The imaging or radionuclide nanocell may be useful in both diagnostic and treatment methods.

All references cited above or below are herein incorporated by reference.

The present invention is further illustrated by the following Examples. Examples are provided to aid in the understanding of the invention and are not construed as a limitation thereof.

### EXAMPLES

### Example 1

The present invention overcomes several limitations of using nanoparticles for imaging, including their insolubility and tendency to aggregate and the general distribution when injected into systemic circulation, which would prevent the discrimination between normal and diseased tissues. Various approaches have been made to keep them stable in suspension including the attachment of pegylated groups, or coating them with various functional groups and peptides for targeted delivery. However, such approaches still fail to overcome the potential for uptake by the reticuloendothelial system (RES) or uptake by normal tissues because of their nanoscale size.

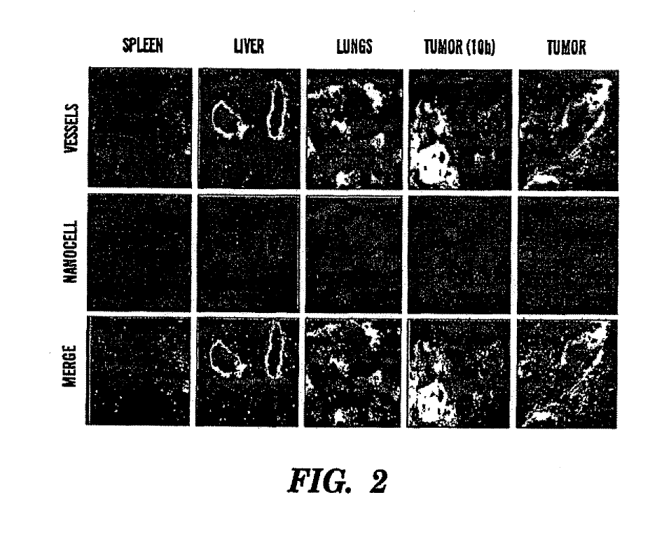

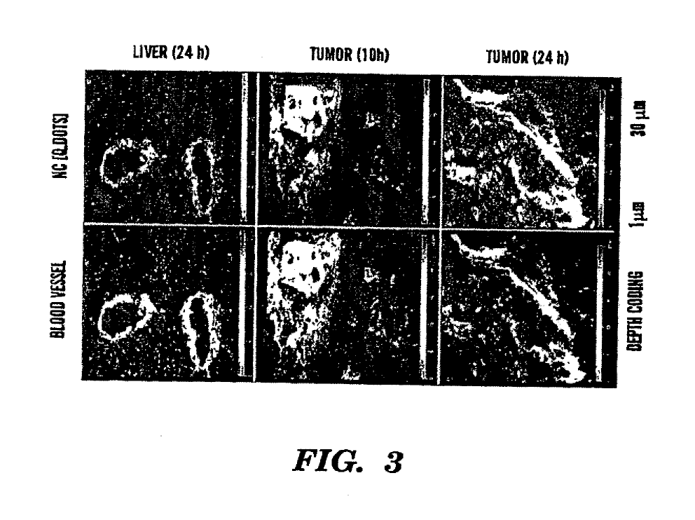

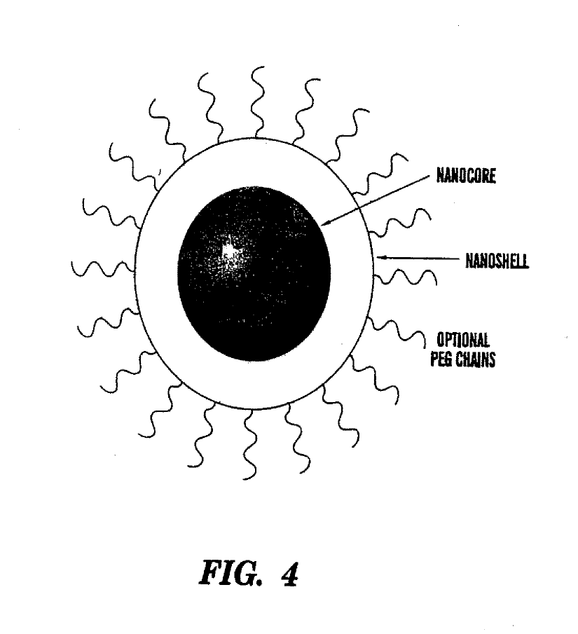

The present invention describes a modified, nuclear nanocell, where the nuclear nanocore is a quantum dot or a nanoparticle that emits a radiation following excitation (FIG. 1). The encapsulation of the nuclear nanocore inside the lipid bilayer, and the presence of the PEG on the surface of the bilayer prevents the RES from recognizing it as a foreign body and therefore the nanocell can escape internalization into normal, non-diseased tissues. Furthermore, the size of the nanocell ranges between 60-600 nm, which is the pore size in tumor vasculature, and therefore the nanocells can extravasate out only from the tumor vasculature and not into any other tissue. This is further supported by the results shown in FIG. 2, where almost no signal from the modified nanocell is detected in spleen (a part of the RES), suggesting that its not taken up by the RES, and is restricted within the vascular component in the liver or lungs, two highly vascular tissues. In contrast, the modified nanocells extravasate out into the solid tumors and show a distinct imaging pattern (FIG. 2, 3). These results indicate that the nuclear nanocells are a powerful imaging technique to identify tumors or other angiogenesis-based diseases.

Results

Localization of Nanocells In Vivo.

Nanocells fabricated with a quantum dot core were injected into tumor-bearing mice. Cross sections of tissues (30.mu.m) harvested at 10 and 24 h post-treatment were immunostained for vWF to delineate the blood vessels. Images were captured using a LSM510 confocal microscope, with excitation at 488 nm and emission for FITC (vWF) and Rhodamine (Qdots).

FIG. 2 shows the staining for vWF, Nanocell and merge images of cross sections of spleen, liver, lungs at 24 hours post-administration, showing that the nanocells are restricted to the vascular compartment. The tumor sections indicate that the nanocells are still within the vasculature at 10 h, and extravasate out by 24 h.

FIG. 3 shows a depth-coding of intensity for vWF and nanocell in a 3D-reconstruction of the tissue sections, which clearly shows that the nanocells extravasate out from the tumor vasculature by 24 h in contrast to physiological vasculature.

Tumor cells were implanted in mice and allowed to grow into solid tumors. The animals were injected with nanocells with a quantum dot core, and sacrificed at 10 h and 24 h post-administration. The tissues were harvested, fixed, and stained for blood vessels. As shown in FIG. 2, there is limited uptake into the spleen, the modified nanocells are restricted in the vasculature of lungs and liver, and the modified nanocells extravagate out in the tumor. The distinction in distribution pattern indicates the modified nanocells usefulness as a diagnostic imaging agent.

Similarly, FIG. 3 shows confocal images of a similar experiment where tumor cells were implanted in mice and allowed to grow into solid tumors. The animals were injected with nanocells with a quantum dot core, and sacrificed at 10 h and 24 h post-administration. The tissues were harvested, fixed, and stained for blood vessels. The images shown in FIG. 3 are depth coding, showing the distribution of the nanocells in a 3-dimension by merging images on the z-axis. As shown in the confocal images, is limited uptake into the spleen, the modified nanocells are restricted in the vasculature of lungs and liver, and the modified nanocells extravagate out in the tumor. The distinction in distribution pattern indicates the modified nanocells usefulness as a diagnostic imaging agent.

Materials and Methods

Synthesis of Nanocells

To prepare the lipid envelope of the nanocell, cholesterol (CHOL), egg-phosphatidylcholine (PC), and distearoylphosphatidylethanolamine-polyethylene glycol (m.w. 2000) (DSPE-PEG) were obtained from Avanti Polar Lipids (Birmingham, Ala.). Combretastatin A4 was obtained from Tocris Cookson (Ellisville, Mo.). All other reagents and solvents were of analytical grade. PC:CHOL:DSPE-PEG (2:1:0.2 molar) lipid membranes were prepared by dissolving 27.5 mg lipid in 2 mL chloroform in a round bottom flask. Combretastatin A4 (12.5 mg) was co-dissolved in the choloroform mixture at a 0.9:1 drug:lipid molar ratio. Chloroform was evaporated using a roto-evaporator to create a monolayer lipid/drug film. This film was resuspended in 1 mL H.sub.20 after one hour of shaking at 65° C. to enable preferential encapsulation of combretastatin A4 within the lipid bilayer. When synthesizing nanocells, nanoparticles containing 250 μg doxorubicin were added to the aqueous lipid resuspension buffer. The resulting suspension was extruded through a 200 nm membrane at 65° C. using a hand held extruder (Avestin, Ottawa, ONT) to create the lipid vesicles. The average vesicle size was determined by dynamic light scattering (Brookhaven Instruments Corp, Holtsville, N.Y.).

Tissue Distribution Studies

Nanocells were fabricated with Quantum Dots in the core, and injected intra-venously into tumor-bearing mice. The animals were sacrificed at different time points, and the highly vascular organs were extracted during necropsy. The tissue sections (30 μm thick) were immuno-stained with an antibody against vonWillebrand factor to delineate the blood vessels. Confocal images were captured at 512×512 resolution, with excitation using a 488 nm laser line and emissions at the F1TC/Rhodamine wavelengths. Depth-coding was done using the LSM510 software.

In Vivo Tumor Model

Male C57/BL6 mice (20 g) were injected with 3×10⁵ GFP-BL6/F10 or 2.5×10⁵ Lewis lung carcinoma cells into the flanks. The growth of the tumors was monitored regularly. The mice were randomized into different treatment groups when the tumor reached 50 mm³ in volume. Each formulation, nanocell or simple liposomes, was prepared, quantified, and diluted such that 100 μl of administration was equivalent to 50 mg/kg and 500 μg/kg of combretastatin and doxorubicin respectively.

Immunohistocytochemistry for Tumor Vasculature

Tumor samples were embedded in TissueTek and snap frozen on dry ice. Thin cryosections (10 μm) were made using a Reichart cryostat, and fixed in methanol. The sections were then permeabilised in Tris buffer saline with Triton X and Tween, and blocked with 1% goat serum. The sections were probed overnight with a rabbit primary antibody against vonWillebrand factor (Dako, 1 in 2000 dilution), an endothelial cell marker. The sections were washed and re-probed with a goat secondary antibody coupled to Texas Red. The sections were coated with slowfade (Molecular probes), and imaged using a Leica LSM510 confocal microscope.

Images were captured randomly from three areas per section. The fluorochromes were excited with 488 nm and 543 nm laser lines, and the images were captured using 505-530 BP and 565-615 BP filters at a 512×512 pixel resolution. Vessel density was quantified using stereological approaches, using a planimetric point-count method using a 224-intersection point square reticulum.

### Example 2

Preparation of Nanocells for Treatment of Asthma

Nanoparticles with dexamethasone were synthesized from PLGA using PVA as a stabilizer using an emulsion-solvent evaporation technique. The nanoparticles were then coated with a shell of lactose using a spray drying technique. The bronchodilator, salbutamol, was dissolved in the lactose solution prior to spray drying. The nanocell formed was then lyophilized overnight before being administered in vivo. For SEM, dehydrated nanoparticles were gold-coated on a carbon grid. They were analyzed using a Jeol EM (magnification, 3700×).

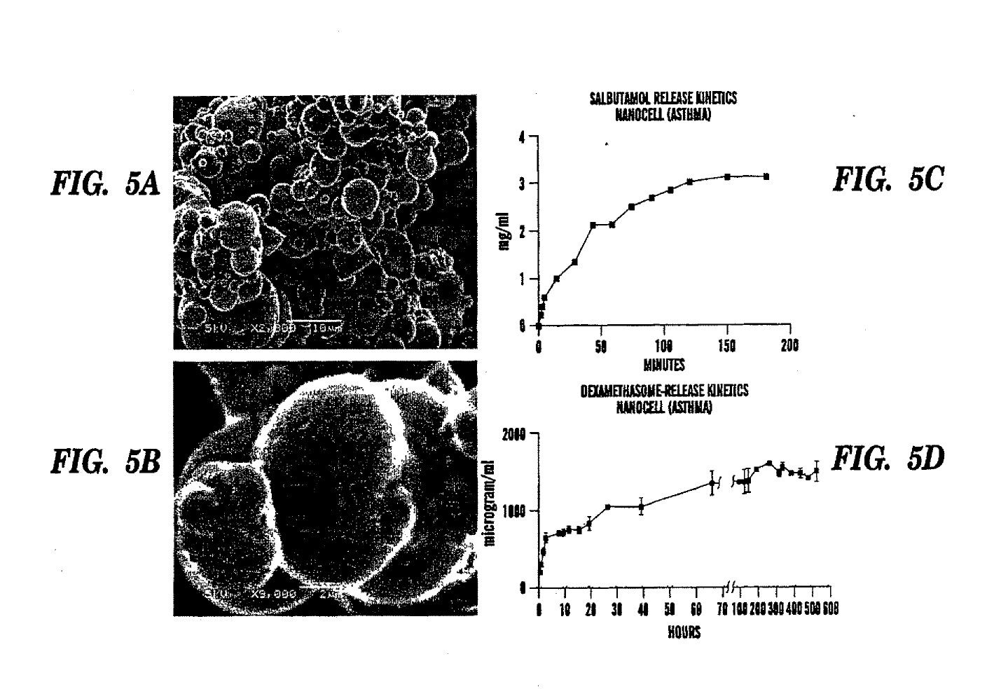

As shown in FIG. 5, electron micrograph revealed that the nanoparticles formed were spherical and were of a diverse size range from 5×10¹-20×10³ nm. The nanoparticles were then coated with a lactose layer, which made the size of the particles in the 10³ to 10⁵ nm range.

Release Kinetics Characterization

Drug-loaded nanocells were suspended in 1 ml of PBS buffer or hypoxic-cell lysate and sealed in a dialysis bag (M.W. cutoff: 10,000). The dialysis bag was incubated in 20 ml of PBS buffer at 37° C. with gentle shaking. Aliquots were extracted from the incubation medium at predetermined time intervals, and released drug was quantified by reverse phase HPLC using a C18 column using a linear gradient of acetonitrile and water eluents.

As shown in FIG. 5, salbutamol is rapidly released from the lactose nanoshell within minutes, reaching a peak concentration within hours. In comparison, the nanocore releases dexamethasone in a delayed manner and the concentration is sustained over hours. This is important as the nanocell thereby enables the rapid relaxation of the constricted airways and delays the release of dexamethasone such that it is available in the lungs right at the time when the delayed chronic inflammation phase starts.

In Vivo Model of Asthma

OVA Sensitization of Rats:

OVA or ovalbumin (Sigma, 1 mg/mL) in PBS was mixed with equal volume of 10% (w/v) aluminum potassium sulfate (alum, Sigma) in deionized water, pH was adjusted to 6.5 using 10 N NaOH and was then incubated in room temperature for 60 minutes. It was then centrifuged at 2000 rpm for 10 minutes and the OVA/alum pellet was resuspended to the original volume in deionized water (1 mg/mL OVA). 32 rats received i.p. injection of 1 mL OVA/alum suspension on day 1.

OVA/alum suspension (10 mg/mL) was made using a similar technique and intratracheal (i.t.) challenges with OVA were performed. In brief, ketamine—xylazine cocktail stock solution was made with 5 mL of ketamine HCl (100 mg/ml) mixed with 0.5 mL xylazine HCl (100 mg/ml). Rats were anesthetized with 0.07 ml/100 grams body weight (administered i.p. and equivalent to 63 mg/kg ketamine and 6 mg/kg xylazine) and were placed on a board in a supine position. OVA/alum suspension (250 μL on day 7 and 125 μL on days 14, 18 and 21) were placed in the back of the tongue. The rats were allowed to recover from the anesthesia after an hour.

Deposition pattern of OVA was examined by toluidine blue dye. OVA/alum (10 mg/mL) suspension was mixed with toluidine blue and 250 μL was administered through the i.t. route. The rat was euthanized after an hour and the respiratory tract and the gastrointestinal tract were dissected out. The toluidine blue staining was visible in the tracheo-bronchial tree, but was not detected in the esophagus and stomach.

OVA Challenge and Treatment:

Rats were divided into the following 8 groups:

Group 1: Control, no OVA challenged, no treatment
Group 2: Control, OVA challenged, no treatment

### Group 3: Free Drug, 100 μg Salbutamol/mg Lactose

### Group 4: Free Drug, 100 μg Dexamethasone/mg Lactose

### Group 5: Free Drug, 100 μg Salbutamol+100 μg Dexamethasone/mg Lactose

### Group 6: Free Drug, 50 μg Salbutamol+100 μg Dexamethasone/mg Lactose

### Group 7: Nanocell Formulation, 100 μg Salbutamol+100 μg Dexamethasone/mg Lactose

### Group 8: Nanocell Formulation, 50 μg Salbutamol+100 μg Dexamethasone/mg Lactose 1

On day 22, rats were anesthetized with i.p. ketamine—xylazine cocktail and respiratory rate and pattern were monitored. Inhalation challenges with 3 mg OVA/rat was performed and rats were monitored for respiratory rate and breathing difficulties following OVA challenge. Group 3-8 rats then received treatment with free or liposome encapsulated nanoparticles (salbutamol and/or dexamethasone) via pulmonary inhalation route. Pulmonary inhalation was completed by using an insufflator (Penn Century, Philadelphia) specially designed for aerosol inhalation in small animals. Rats were then observed for respiratory rates and response to treatment.

### Sample Collection:

Six hours following administration of treatment, anesthetized rats were euthanized by cardiac puncture. Blood samples were collected for blood cell count. The respiratory tract of the animal was dissected out. Broncho-alveolar lavage (BAL with 1.5 mL saline, three times) was collected for cytopathology and markers of asthma from the right lung after tying off the left lung in the main-stream bronchus. The trachea and upper and lower lobes of the left lung was collected and preserved in 10% formalin for histopathology.

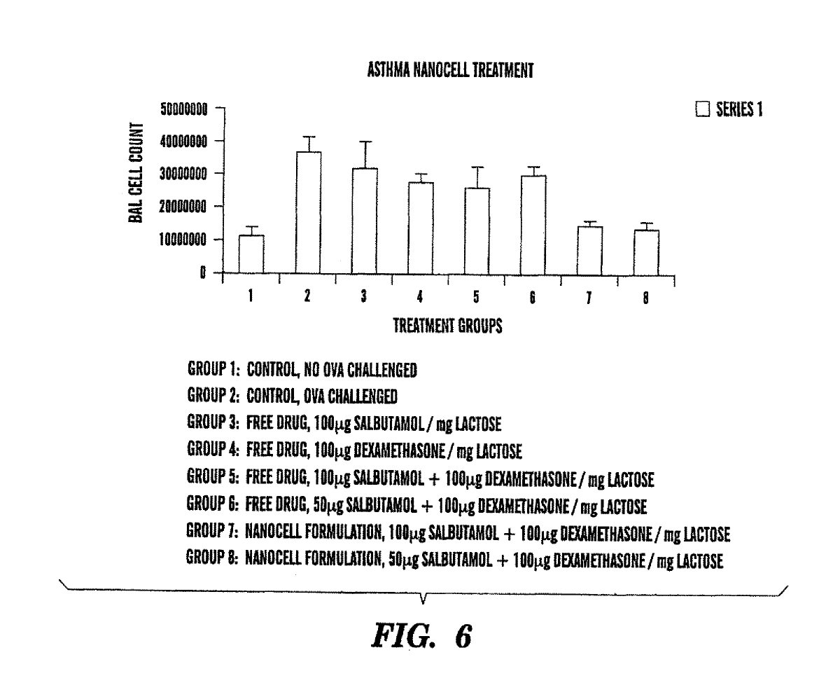

As shown in FIG. 6, treatment with nanocells keep the level of infiltrated cells in the lungs of ova-challenged mice comparable to the level seen in unchallenged normal mice. In contrast, a simple addition of the dexamethasone and salbutamol was unable to reduce the inflammation to the basal level. This indicates that the delayed release of the corticosteroid from the nanocore ensures less drug is being absorbed into the blood circulation and most of it is available for activity in the lungs after 6 hours, i.e. when the inflammatory stage starts. In contrast, most of the drug is absorbed when administered free and less is available within the lungs for inhibiting inflammation.

### Example 3

Despite major advances in the development in anticancer drugs and imaging agents, a major disadvantage is their lack of selectivity for malignant tissue. Currently, most common drug delivery systems and imaging agents target proteins that are overexpressed on the surface of cancer cells. Alterations to the normal function of the glycosylation machinery have been increasingly recognized as a consistent indication of malignant transformations and tumorigenesis. In many cases, these alterations result in the overexpression of specific cancer-associated carbohydrates, specifically on the malignant tissue. Due to the complexity of molecular interactions with carbohydrates, very few systems have been designed to specifically target carbohydrates for imaging and drug delivery purposes. Despite the use of lectins for detection of carbohydrates in different tissues, their low affinity, high molecular weight, the stability of their active structures and their complexity for selective chemical modifications has limited their use for medicinal applications. Therefore, new systems are needed to improve the selective delivery of imaging and therapeutic agents to disease tissue. In this example, we show that synthetic conjugates serve as reliable systems for this urgently-needed endeavor. These molecular delivery systems are of important value to the biotechnology/pharmaceutical/diagnostic industry as new formulations of therapeutic agents or imaging systems.

This example shows a designed and synthesized molecular scaffolds that targets cancer-associated carbohydrates in different tissues. Specifically, we use nano-scale scaffolds to display the carbohydrate-binding molecules in multivalent fashion in order to increase the selectivity and affinity of the conjugates to the cancer-associated carbohydrate. These scaffolds are conjugated to different imaging probes in order visualize the selectivity of our conjugates for malignant tissue. As a primary screening method for binding and selectivity we have used tissue arrays that contain a wide variety of different cancerous tissue in addition to their match controls. Our results show that the synthetic conjugates display good selectivity and sensitivity to specific cancerous tissue over non-malignant tissue. We have also tested these conjugates in animal models and have shown increased localization in tumors. These synthetic conjugates can also be derivatized with different drugs for the selective delivery of therapeutics to diseased tissue.

### Results

Transformations on the structures of mammalian cell-surface carbohydrates can lead to pathologic alterations in cellular adhesion and motility functions, ultimately leading to carcinoma cell aggregation and metastasis. Examples of these alterations have been observed in colon cancer mucins, the major glycoprotein constituents of the protective mucus on the colon's epithelial surface. These carbohydrate-rich epithelial glycoproteins are described in terms of core type, backbone type, and peripheral structures; and the differences in these structures are currently under investigation for diagnostic and prognostic markers. Many cancer-associated mucins typically show increases in core type 1, Thomsen-Friedenreich antigen (TF antigen), an immunodominant Galβ1-3GalNAca disaccharide that is found sialylated on normal cells but nonsialylated in carcinoma cells.

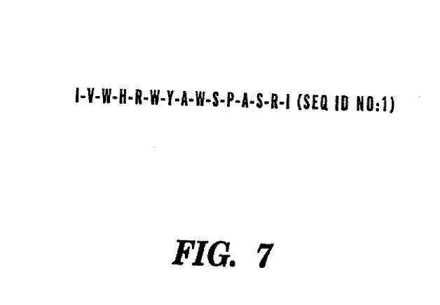

Despite the use of peanut agglutinin (PNA) lectin (and other lectins) for detection of the TF antigen in different tissue samples, its low affinity, high molecular weight, the stability of its active structure and its complexity for selective chemical modifications has limited its use for medicinal applications. Recently, a peptide with good affinity and selectivity towards the TF antigen has been selected from phage display libraries (FIG. 7).^{1.2} The stability and numerous possible accessible chemical modifications have opened new avenues to use this TF antigen-targeting agent for different clinical applications. However, it is now known that the selectivity and affinity of carbohydrate-binding partners for their antigen is highly dependent on valency. In fact, this peptide binds the TF antigen with 0.6 μM affinity when displayed as a monomer. As one of our major goals is the selective targeting of cancerous tissue, increasing the affinity and selectivity of the targeting agent is essential. Nanoscale scaffolds provide a large surface area that allows multiple sites for derivatization with targeting agents. Herein, we take advantage of the surface provided by nanoscale scaffolds to display carbohydrate-binding partners in multivalent fashion as a way to optimize selectivity and affinity of the targeting agent for cancerous tissue. In the context of the nanocell, the targeting agent is preferentially incorporated onto the external surface of the nanoshell but can also be incorporated onto the surface of the inner core of the nanocell.

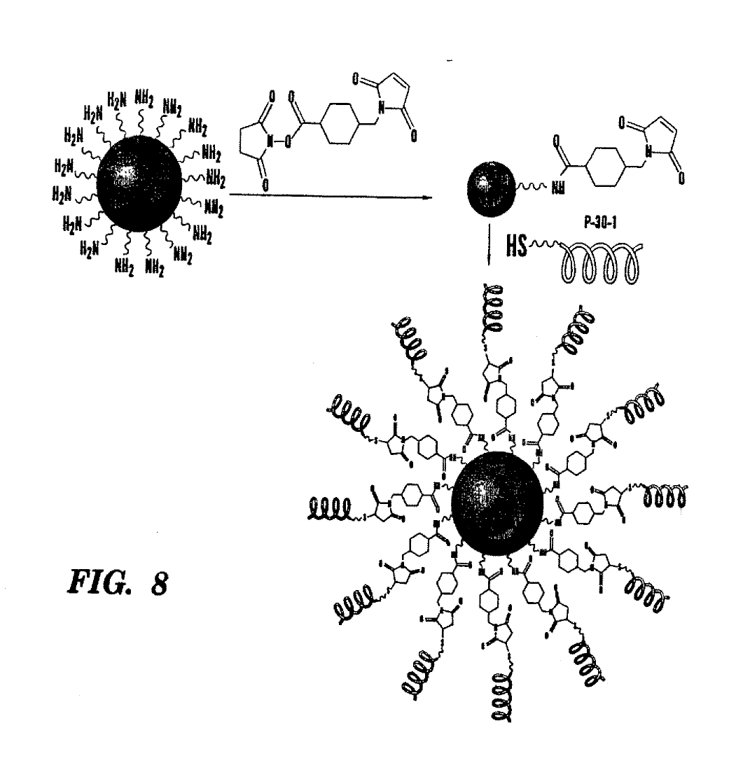

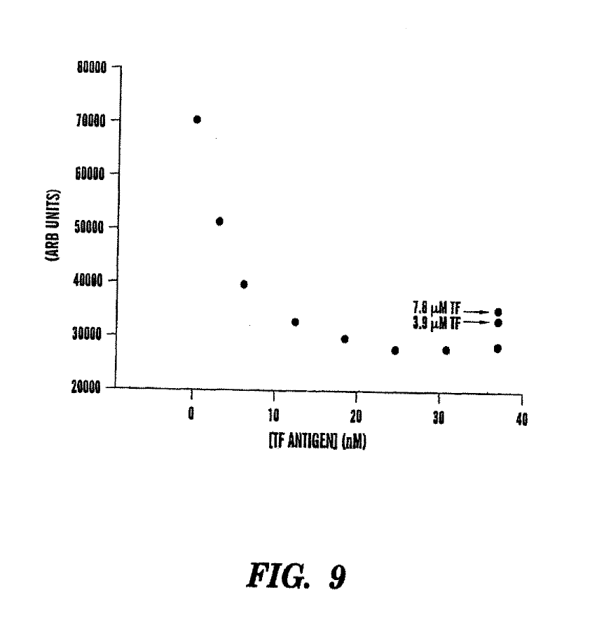

As an example, herein, we have used semiconductor nanocrystals (quantum dots) to display synthetic peptides on a multivalent fashion to selectively target cancer-associated carbohydrates on the surface of cancer cells. In this example, the TF antigen-binding peptide described above was modified to incorporate a thiol functional group at the N-terminus for selective conjugation to maleimides inserted at the end of the polyethylene glycol (PEG) spacers on the surface of the nanocrystals. The PEG spacers between the quantum dot and the peptide increase the flexibility of the peptide and therefore facilitate the multivalent interaction with their antigen on cell surfaces (FIG. 8). When tested for specificity and affinity to bind the TF antigen via fluorescent energy transfer (FRET) experiments, the nanocrystal conjugate showed specific binding an enhanced affinity (approximately 3 nM). FIG. 9, shows the quenching of the quantum dot emission at 565 nm via FRET mechanism by fluorescently-labeled asialofetuin (which contains the TF antigen). As shown in the figure, the discosiation of the nanocrystal-asialofetuin complex by the addition of the free TF antigen demonstrate the specificity of the interaction.

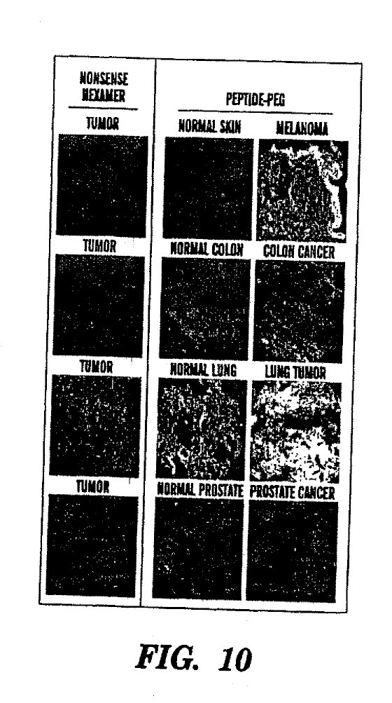

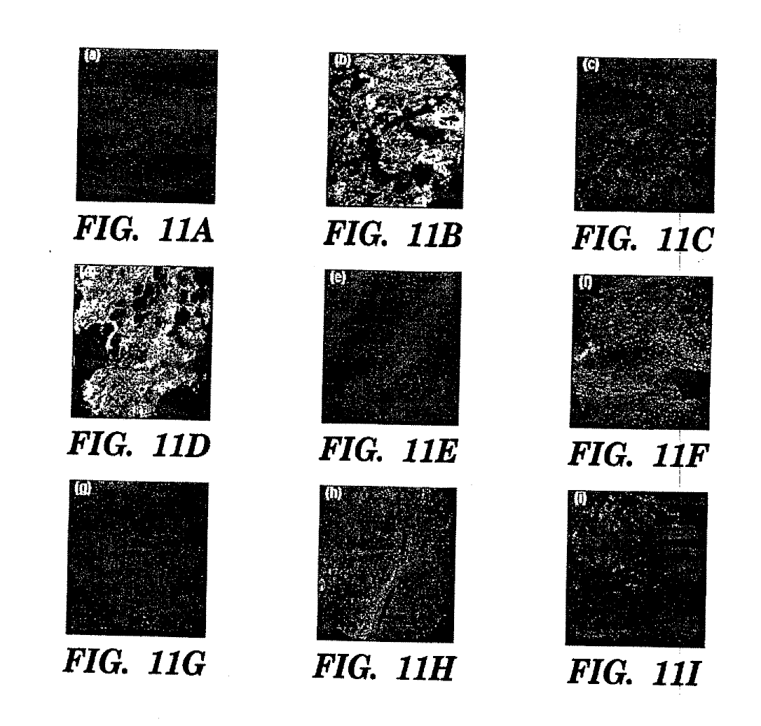

Using tissue array systems, we efficiently scanned the selectivity of these nano-scale scaffolds for different human tissues. As a control, we also derivatized the nanocrystals with a random peptide sequence of six amino acids (6mer). FIG. 10 shows the contrast in selectivity of the TF antigen-binding conjugate for cancerous tissue in comparison to the hexamer conjugate. The TF antigen-binding conjugate especially showed specific binding towards lung cancer, melanoma and non-hodgkin's lymphoma (FIG. 11).

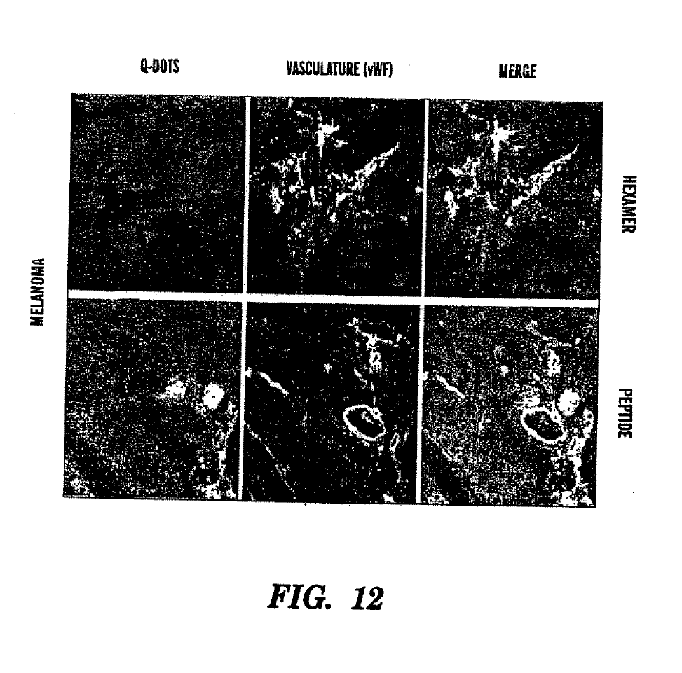

In order to evaluate the selectivity of the conjugates in vivo we tested these in a melanoma mouse model. The conjugates were injected into the tumor-bearing mice and cross sections of tissues (30 μm) harvested at 24 h post-treatment were analyzed using a LSM510 confocal microscope. As shown in FIG. 12, accumulation of the quantum dots in the tumor was observed for the TF antigen-binding conjugate but not for the hexamer conjugate. This confirms the selectivity of the carbohydrate targeting agent for cancerous tissue.

### Methods

### Peptide Synthesis

The peptides (PrPUP) were synthesized on PAL-PEG-PS resin by using an automated ACT peptide synthesizer. The peptides were prepared as the C-terminal amide and the N-terminal acetyl derivative. Standard 9-fluorenylmethoxycarbonyl (Fmoc) chemistry and HBTU/HOBT activation was used for all residues except cysteine. In this case, preactivated Fmoc-L-Cys(Trt)-OPfp was used in the absence of base to prevent racemization.

Peptide Purification and Characterization

Peptides were dissolved in 85:10:5 cold H₂O/CH₃CN/DMSO (with 0.1% trifluoroacetic acid, TFA), filtered through a 0.45-μm filter and purified by reverse-phase HPLC on a Waters Prep LC 4000 system using a 5-60% gradient in acetonitrile/0.1% TFA for 30 min. Peptides were collected and characterized by electrospray mass spectrometry (ESMS). TF antigen-binding peptide: [[M+3H⁺]/3 691.4 (observed); 691.8 (calculated)] and hexameter peptide: [[M+H⁺] 774.5 (observed); 774.9 (calculated)].

Quantum Dots Derivatization with Carbohydrate-Binding Peptides:

Quantum dots (565 nm) were obtained from Quantum Dot Corporation/Invitrogen (Hayward, Calif.). The quantum dots contain a 2,000 molecular weight PEG spacer covalently attached to the surface of the nano-particle and a primary amine on the other side of the PEG spacer. The peptide was attached using the standard protocols for antibodies provided by the quantum dot supplier. Briefly, the amines on the surface of the quantum dots are first modified using the hetero-bifunctional crosslinker 4-(maleimidomethyl)-1-cyclohexanecarboxylic acid N-hydroxysuccinimide ester (SMCC) followed by reacting the maleimides with the terminal cysteine thiol on the peptide.

### Tissue Binding Studies:

Tissue binding studies were performed on a Amicon TMA 1010 tissue array containing different cancer tissues and normal controls. Samples were incubated with tissues for 4 hours and after washing the unbound molecules, the tissues were analyzed using a LSM510 confocal microscope.

### REFERENCES

1. Landon, L. A. et al. Combinatorial evolution of high-affinity peptides that bind to the Thomsen-Friedenreich carcinoma antigen. J Protein Chem 22, 193-204 (2003).
2. Landon, L. A., Zou, J. & Deutscher, S. L. Effective combinatorial strategy to increase affinity of carbohydrate binding by peptides. Mol Divers 8, 35-50 (2004).
   All references described herein are incorporated by reference in their entirety.

We claim:

1. A radionuclide-nanocell composition comprising:
a nanocell having an inner nanocore bound to a ligand that will bind to a radionuclide, and an outer layer comprising lipid and polyacetylene glycol, wherein the radionuclide forms a complex with the ligand bound to the inner nanocore, wherein the nanocell is less than 600 nm in diameter.

2. The radionuclide-nanocell composition of claim 1, wherein the radionuclide is selected from the group consisting of (99m)Tc, (95)Tc, (111)In, (62)Cu, (64) Cu, (67)Ga, and (68)Ga, Iodine-123, Iodine-131, Ruthenium-97, Copper-67, Cobalt-57, Cobalt-58, Chromium-51, Iron-59, Selenium-75, Thallium-201, Tc, Ru, Co, Pt, Fe, Os, Ir, W, Re, Cr, Mo, Mn, Ni, Rh, Pd, Nb, Ta, and Ytterbium-169.

3. The radionuclide-nanocell composition of claim 1, further comprising a targeting ligand.

4. The radionuclide-nanocell composition of claim 1, further comprising a therapeutic moiety.

5. The radionuclide-nanocell composition of claim 1, wherein the polyacetylene glycol is polyethylene glygol (PEG).

6. A method for in vivo detection of an angiogenic or a malignant tissue comprising:
a) administering to an individual the radionuclide-nanocell composition of claim 1, wherein the nanocell is about 60 to about 600 nm in total diameter; and
b) imaging the individual after a period of time, wherein the period of time is a time when the radionuclide-nanocell composition has had time to enter a tissue, wherein the presence of the radionuclide in the tissue indicates that the tissue is angiogenic or malignant.

7. The method of claim 6 wherein the angiogenic tissue is a result of an angiogenic disease or disorder, or malignancy selected from the group consisting of cancer, solid tumor or solid tumor metastasis, retinopathy, diabetic retinopathy, macular degeneration, hemangioma, ulcerative colitis, Crohn's disease, osteoarthritis, rheumatoid arthritis, corneal graft rejection, neovascular glaucoma and retrolental fibroplasia.

8. A nanocell composition comprising a nanocell having an inner nanocore which excites and emits defined wavelengths, encapsulated within an outer nanoshell comprising lipid and polyacetylylene glycol, wherein the nanocell is less than 600 nm in diameter.

9. The nanocell composition of claim 8, wherein the nanocore is selected from a quantum dot, nanowire, nanotube or a fluorochrome-coupled nanoparticle.

10. The nanocell composition of claim 8, further comprising a targeting ligand.

11. The nanocell composition of claim 8, further comprising a therapeutic moiety.

12. The nanocell composition of claim 8, wherein the polyacetylene glycol is polyethylene glygol (PEG).

13. The nanocell composition of claim 8, wherein the inner nanocore is associated with at least one first therapeutic and the outer nanoshell is associated with at least one second therapeutic, wherein the outer nanoshell and inner nanocore are formulated to release the first therapeutic and the second therapeutic at a different rate.

14. The nanocell composition of claim 13, further comprising at least one targeting ligand.

15. The nanocell composition of claim 13, wherein the first therapeutic differs from the second therapeutic.

16. The nanocell composition of claim 13, wherein the first therapeutic is the same as the second therapeutic.

17. The nanocell composition of claim 13, wherein the nanocell size is greater than about 60 nm in diameter.

18. The nanocell composition of claim 13, wherein the first therapeutic is selected from the group consisting of a corticosteroid, iopanoic acid, radioiodine, an antibiotic, and an antifribrotic.

19. The nanocell composition of claim 13, wherein the second therapeutic is selected from the group consisting of a chemotherapeutic agent, a bronchodilator, an antithyroid drug, a beta-blocker, a recombinant human deoxyribonuclease (rhDNase), and a corticosteroid.

20. A method for in vivo treatment of angiogenic diseases, disorders, or malignancy comprising: administering to an individual the tailored nanocell composition of claim 13, wherein the nanocell is about 60 to about 600 nm in total diameter, and wherein the first therapeutic is an anti-neoplastic and the second therapeutic is anti-angiogenic.

21. The method of claim 20 wherein the angiogenic disease, disorder, or malignancy is selected from the group consisting of cancer, solid tumor or solid tumor metastasis, retinopathy, diabetic retinopathy, macular degeneration, hemangioma, ulcerative colitis, Crohn's disease, osteoarthritis, rheumatoid arthritis, corneal graft rejection, neovascular glaucoma and retrolental fibroplasia.

22. A method for treatment of a disease or disorder comprising administering to a subject in need thereof the tailored nanocell composition of claim 13.

23. The method of claim 23, wherein the disease or disorder is selected from the group consisting of brain tumors, asthma, Grave's disease, cystic fibrosis, pulmonary fibrosis, and an angiogenic disease.


SEQUENCE LISTING (1 sequence)

SEQ ID NO: 1 - protein, 15 aa, Artificial Sequence
  Description of Artificial Sequence Synthetic peptide
IVWHRWYAWSPASRI